(19) United States
(12) Patent Application Publication
(10) Pub. No.: US 2012/0177593 A1
(43) Pub. Date: Jul. 12, 2012

(54) SYNTHESIS OF DENDRIMER CONJUGATES

(75) Inventors: James R. Baker, JR., Ann Arbor, MI (US); Yuehua Zhang, Ann Arbor, MI (US); Thommey P. Thomas, Dexter, MI (US); Ankur Mahesh Desai, Ann Arbor, MI (US)

(73) Assignee: THE REGENTS OF THE UNIVERSITY OF MICHIGAN, Ann Arbor, MI (US)

(21) Appl. No.: 13/383,378

(22) PCT Filed: Jul. 20, 2010
(86) PCT No.: PCT/US2010/042556

§ 371 Date: Mar. 26, 2012

Related U.S. Application Data
(60) Provisional application No. 61/226,993, filed on Jul. 20, 2009.

(51) Int. Cl. A61K 31/785; A61P 19/02; A61P 35/00; C08G 73/02; G01N 33/53

(52) U.S. Cl. 424/78.17; 525/426; 435/7.2

(57) ABSTRACT

The present invention relates to novel methods of synthesis of therapeutic and diagnostic dendrimers. In particular, the present invention is directed to novel dendrimer conjugates, novel methods of synthesizing the same, compositions comprising the conjugates, as well as systems and methods utilizing the conjugates (e.g., in diagnostic and/or therapeutic settings (e.g., for the delivery of therapeutics, imaging, and/or targeting agents (e.g., in disease (e.g., cancer, inflammatory disease) diagnosis and/or therapy, pain therapy, etc.)). Accordingly, dendrimer conjugates of the present invention may further comprise at least two different components for targeting, imaging, sensing, and/or providing a therapeutic or diagnostic material and/or monitoring response to therapy. Furthermore, the novel synthesis methods of certain embodiments of the present invention provide significant advantages with regard to total reaction time and simplicity.

### CROSS REFERENCE TO RELATED APPLICATION

This application claims priority to U.S. Provisional Patent Application Ser. No. 61/226,993, filed Jul. 20, 2009, hereby incorporated by reference in its entirety.

### STATEMENT REGARDING FEDERALLY SPONSORED RESEARCH OR DEVELOPMENT

This invention was made with government support under Grant No. 1 R01 CA119409 awarded by the National Institutes of Health. The government has certain rights in the invention.

### FIELD OF THE INVENTION

The present invention relates to novel methods of synthesis of therapeutic and diagnostic dendrimers. In particular, the present invention is directed to novel dendrimer conjugates, methods of synthesizing the same, compositions comprising the conjugates, as well as systems and methods utilizing the conjugates (e.g., in diagnostic and/or therapeutic settings (e.g., for the delivery of therapeutics, imaging, and/or targeting agents (e.g., in disease (e.g., cancer) diagnosis and/or therapy, pain therapy, etc.))). Accordingly, dendrimer conjugates of the present invention may further comprise at least two different components for targeting, imaging, sensing, and/or providing a therapeutic or diagnostic material and/or monitoring response to therapy. Furthermore, the novel synthesis methods of certain embodiments of the present invention provide significant advantages with regard to total reaction time and simplicity.

### BACKGROUND OF THE INVENTION

Cancer remains the number two cause of mortality in the United States, resulting in over 500,000 deaths per year. Despite advances in detection and treatment, cancer mortality remains high. New compositions and methods for the imaging and treatment (e.g., therapeutic) of cancer may help to reduce the rate of mortality associated with cancer.

Severe, chronic pain is observed a variety of subjects. For example, there exist large numbers of individuals with severe pain associated with arthritis, autoimmune disease, injury, cancer, and a host of other conditions.

A vast number of different types of pain medications exist. For example, a number natural and synthetic alkaloids of opium (i.e., opioids) are useful as analgesics for the treatment of severe pain. However, a number of severe side effects associated with opioid and other pain medication usage exist. For example, administration of opioid agonists often results in intestinal dysfunction due to action of the opioid agonist upon the large number of receptors in the intestinal wall. Opioids are generally known to cause nausea and vomiting as well as inhibition of normal propulsive gastrointestinal function in animals, resulting in side effects such as constipation.

Pain medication (e.g., opioid)-induced side effects are a serious problem for patients being administered pain medications (e.g., opioid analgesics) for both short term and long term pain management. For instance, more than 250,000 terminal cancer patients each year take opioids, such as morphine, for pain relief, and about half of those patients experience severe constipation. At present, patients receiving opioid pain medications face the difficult choice of suffering burdensome adverse effects (e.g., constipation) or ineffective analgesia.

There exists a need for compositions, methods and systems for delivering agents (e.g., diagnostic and/or therapeutic (e.g., cancer and/or pain therapeutics) to subjects that provide effective therapy (e.g., disease treatment, symptom relief, etc.) with reduced or eliminated side effects, even when administered in high doses. Functionalized dendrimers, such as PAMAM dendrimers conjugated to functional ligands relevant to cancer therapy and/or pain alleviation, have been developed for such purposes. However, classical synthesis methods of ligand-conjugated PAMAM dendrimers have limitations in terms of time required for the synthesis method and complexity of the synthesis scheme.

### SUMMARY

The present invention relates to novel methods of synthesis of therapeutic and diagnostic dendrimers. In particular, certain embodiments of the present invention encompass novel dendrimer conjugates, methods of synthesizing the same, compositions comprising the conjugates, as well as systems and methods utilizing the conjugates (e.g., in diagnostic and/or therapeutic settings (e.g., for the delivery of therapeutics, imaging, and/or targeting agents (e.g., in disease (e.g., cancer) diagnosis and/or therapy, pain therapy, etc.)). Accordingly, in some embodiments, dendrimer conjugates of the present invention may further comprise one or more components for targeting, imaging, sensing, and/or providing a therapeutic or diagnostic material and/or monitoring response to therapy. Furthermore, the novel synthesis methods of certain embodiments of the present invention provide significant advantages with regard to total reaction time, yield, purity, energetic requirements, ability to tune the reaction (e.g., for desired numbers or proportions of different ligands), and simplicity.

Functionalized dendrimers, such as PAMAM dendrimers conjugated to functional therapeutic, targeting, trigger, or imaging ligands, have been developed. However, classical synthesis methods of ligand-conjugated PAMAM dendrimers (e.g., dendrimers conjugated with functional groups) have limitations in terms of time required for the synthesis method and complexity of the synthesis scheme. Previous conjugation of ligands (e.g., functional groups) to dendrimers involved a multiple step synthetic route involving, for example, amide and ester linkages (see, e.g., Kukowska-Latallo, J. F., et al., (2005) Cancer Res 65, 5317-5324; Quintana, A., et al., (2002) Pharmaceutical Research 19, 1310-1316; Majoros, I. J., et al., J Med Chem 48, 5892-5899; each herein incorporated by reference in their entireties). For example, the synthetic steps involved partial acetylation of the dendrimer, conjugation of ligand (e.g., functional group) using EDC chemistry through amide bonds, glycidation of the remaining amino groups, and finally conjugation of additional ligands (e.g., additional functional groups) through ester linkage through some of the glycidol moieties. The variability in efficiency of each of these synthetic steps resulted in batch-to-batch reproducibility issues which limited the application of this technology.

The present invention overcomes such synthetic limitations through providing simiplified methods for synthesizing conjugated dendrimers. In particular, the present invention provides methods for synthesizing multifunctional dendrimers (e.g., dendrimers conjugated with one or more functional groups) through, for example, initial glycidation of a dendrimer followed by simultaneous conjugation of one or more functional groups (e.g., through ester linkages in a “one pot” reaction). The novel methods of the present invention represent a significant improvement over previous synthetic methods in terms of, for example, lower total reaction time, higher yield, and greater ease of manufacturing. In addition, in one non-limiting example, a dendrimer-FA-MTX (PAMAM dendrimer/folic acid/methotrexate) conjugate synthesized by the novel methods of the present invention displayed similar cytotoxic potency as compared to dendrimer-FA-MTX conjugates that had been synthesized using alternative synthetic approaches. The methods are not limited by the nature of the ligand, the nature of the dendrimer, or the nature of the one-pot synthesis reaction.

Accordingly, in certain embodiments, the present invention provides methods for synthesizing dendrimer conjugates (e.g., dendrimers conjugated with one or more functional groups) through, for example, initial glycidolation of a dendrimer molecule (e.g., such that the terminal dendrimer molecule is rendered with terminal hydroxyl groups instead of terminal NH₂ groups), and conjugation of one or more functional groups (e.g., therapeutic agents, targeting agents, trigger agents, and imaging agents) with the glycidolated dendrimer molecule.

The methods are not limited to a particular manner of dendrimer glycidation. In some embodiments, dendrimer glycidation involves exposure and mixing of dendrimer molecules with glycidol.

The methods are not limited to a particular manner of conjugating the functional groups with the glycidated dendrimer molecule. In some embodiments, the conjugation involves ester linkage between a terminal hydroxyl group on the glycidated dendrimer and the functional group. In some embodiments, the conjugation of the one or more functional groups occurs simultaneously (e.g., two or more different functional groups are simultaneously exposed to the glycidolated dendrimer). In some embodiments, the conjugation occurs via a one-pot synthesis reaction. As noted, methods for synthesizing dendrimer conjugates through such techniques (e.g., initial glycidolation of a dendrimer followed by simultaneous conjugation of one or more functional groups (e.g., through ester linkages in a one-pot reaction)) results in, in comparison to previous synthetic methods, lower total reaction time, higher dendrimer conjugate yield, and greater ease of manufacturing.

The present invention is not limited to a particular one-pot synthesis technique. In certain embodiments, the one-pot synthesis reaction occurs by combining all reactants in a single reaction vessel. Reactants may be added simultaneously or sequentially. The method is not limited by the order of addition of reactants, nor by the amount of time passing between any sequential addition of reactants. The reaction is not limited by the relative proportion of reactants. In certain embodiments, it is possible to tune the reaction by altering the relative proportion of reactions. In one non-limiting example, the reaction feed molar ratio of different ligands may be altered to affect the average number and relative proportion of ligands attached to the dendrimer. In certain embodiments where two different ligands are attached to a dendrimer (e.g. a glycidolated G5 PAMAM dendrimer), where the reaction feed molar ratio is represented as A:B:C where A is the relative molarity of ligand 1, B is the relative molarity of ligand 2, and C is the relative molarity of glycidolated G5 PAMAM dendrimer, the value of each of A, B, and C may be varied from 1 to 100. In preferred embodiments, the value of C is held at 1 and the values of each of A and B range from 1 to 50.

The one-pot synthesis reaction method is not limited by the size or shape of the vessel in which it is performed or the material from which the vessel is made. The reaction is not limited by reaction volume. Volume of the reaction may be less than 5 ml, 5-10 ml, 10-20 ml, 20-50 ml, 50-100 ml, 100-1000 ml, 1 L-25 L, 25-50 L, 50 L or more. The reaction is not limited by the pressure at which it is performed. In preferred embodiments, the reaction is conducted at atmospheric pressure. In some embodiments, the reaction occurs under an inert gas. In preferred embodiments, the inert gas is N₂. The reaction is not limited by the temperature at which it is conducted. In preferred embodiments, the reaction is performed at room temperature, e.g. at a temperature ranging from approximately 19-27° C. In particularly preferred embodiments, room temperature is approximately 22° C. The reaction is not limited by the duration of reaction time. Reaction time may be less than 1 hour, 1-5 hours, 5-10 hours, 10-20 hours, 20-30 hours, 30 hours or more. In preferred embodiments, the reaction occurs for 24 hours. In preferred embodiments, the reaction occurs in a suitable solvent system. Suitable solvent systems include but are not limited to polar solvent systems. Examples of polar solvent systems include but are not limited to dimethylsulfoxide (DMSO); N,N-dimethylformamide; N-N-dimethylacetamide; 2-pyrrolidinone; 1-methyl-2-pyrrolidinone; dioxane or any combination thereof.

In addition, the present invention provides novel analytical approaches for calculating molecules of functional groups (e.g., folic acid and methotrexate) attached to a particular dendrimer molecule through, for example, combining characterization techniques of ¹H NMR and MALDI-TOF.

In certain embodiments, the formation of ester bonds (e.g., between a functional group and a terminal hydroxyl group on a glycidolated dendrimer) is facilitated by the presence of ester coupling agents. Examples of ester coupling agents include but are not limited to 2-chloro-1-methylpyridinium iodide and 4-(dimethylamino)pyridine, or dicyclohexylcarbodiimide and 4-(dimethylamino)pyridine or diethyl azodicarboxylate and triphenylphosphine or other carbodiimide coupling agent and 4-(dimethylamino)pyridine.

In certain embodiments, the present invention provides compositions comprising a dendrimer generated through such methods (e.g., a dendrimer one or more ligands (e.g., functional groups) attached to the dendrimer by an ester bond). In certain embodiments, the dendrimer composition is purified prior to inclusion in additional reactions, prior to analysis, or prior to final use. Purification methods include but are not limited to dialysis, precipitation, and chromatographic separation. As non-limiting examples, purification may occur by dialysis against water, or dialysis against buffer, or dialysis against isotonic saline solution, or against any sequential combination of dialysis solutions (e.g., buffer and then water, isotonic saline solution and then water). As further non-limiting examples, purification may occur by precipitation in organic solvents such as diethyl ether, hexane, cyclohexane, ethyl acetate, acetone, chloroform, dichloromethane, tetrahydrofuran, or any combination solution of aforementioned solvents, or any combination solution of aforementioned solvents and more polar solvents such as dioxane, ethanol, methanol, N,N-dimethylformamide, dimethylsulfoxide, N,N-dimethylacetamide, 2-pyrrolidinone, and 1-methyl-2-pyrrolidinone.

The present invention is not limited to particular ligand types (e.g., functional groups) (e.g., for conjugation with dendrimers). Examples of ligand types (e.g., functional groups) include but are not limited to therapeutic agents, targeting agents, trigger agents, and imaging agents.

In some embodiments, the ligand(s) (e.g., functional group(s)) is attached with the dendrimer via a linker. The present invention is not limited to a particular type or kind of linker. In some embodiments, the linker comprises a spacer comprising between 1 and 8 straight or branched carbon chains. In some embodiments, the straight or branched carbon chains are unsubstituted. In some embodiments, the straight or branched carbon chains are substituted with alkyls.

Examples of therapeutic agents include, but are not limited to, a chemotherapeutic agent, an anti-oncogenic agent, an anti-angiogenic agent, a tumor suppressor agent, an anti-microbial agent, an expression construct comprising a nucleic acid encoding a therapeutic protein, a pain relief agent, a pain relief agent antagonist, an agent designed to treat an inflammatory disorder, an agent designed to treat an autoimmune disorder, an agent designed to treat inflammatory bowel disease, and an agent designed to treat inflammatory pelvic disease. In some embodiments, the agent designed to treat an inflammatory disorder includes, but is not limited to, an antirheumatic drug, a biologicals agent, a nonsteroidal anti-inflammatory drug, an analgesic, an immunomodulator, a glucocorticoid, a TNF-a inhibitor, an IL-1 inhibitor, and a metalloprotease inhibitor. In some embodiments, the antirheumatic drug includes, but is not limited to, leflunomide, methotrexate, sulfasalazine, and hydroxychloroquine. Examples of biologicals agents include, but are not limited to, rituximab, finfliximab, etanercept, adalimumab, and golimumab. In some embodiments, the nonsteroidal anti-inflammatory drug includes, but is not limited to, ibuprofen, celecoxib, ketoprofen, naproxen, piroxicam, and diclofenac. In some embodiments, the analgesic includes, but is not limited to, acetaminophen, and tramadol. In some embodiments, the immunomodulator includes but is not limited to anakinra, and abatacept. In some embodiments, the glucocorticoid includes, but is not limited to, prednisone, and methylprednisone. In some embodiments, the TNF-a inhibitor includes but is not limited to adalimumab, certolizumab pegol, etanercept, golimumab, and infliximab. In some embodiments, the autoimmune disorder and/or inflammatory disorder includes, but is not limited to, arthritis, psoriasis, lupus erythematosus, Crohn's disease, and sarcoidosis. In some embodiments, examples of arthritis include, but are not limited to, osteoarthritis, rheumatoid arthritis, septic arthritis, gout and pseudo-gout, juvenile idiopathic arthritis, psoriatic arthritis, Still's disease, and ankylosing spondylitis.

Ligands suitable for use in certain method embodiments of the present invention are not limited to a particular type or kind of targeting agent. In some embodiments, the targeting agent is configured to target the composition to cells experiencing inflammation (e.g., arthritic cells). In some embodiments, the targeting agent is configured to target the composition to cancer cells. In some embodiments, the targeting agent comprises folic acid. In some embodiments, the targeting agent binds a receptor selected from the group consisting of CFTR, EGFR, estrogen receptor, FGR2, folate receptor, IL-2 receptor, VEGFR. In some embodiments, the targeting agent comprises an antibody that binds to a polypeptide selected from the group consisting of p53, Muc1, a mutated version of p53 that is present in breast cancer, HER-2, T and Tn haptens in glycoproteins of human breast carcinoma, and MSA breast carcinoma glycoprotein. In some embodiments, the targeting agent comprises an antibody selected from the group consisting of human carcinoma antigen, TP1 and TP3 antigens from osteocarcinoma cells, Thomsen-Friedenreich (TF) antigen from adenocarcinoma cells, KC-4 antigen from human prostrate adenocarcinoma, human colorectal cancer antigen, CA125 antigen from cystadenocarcinoma, DF3 antigen from human breast carcinoma, and p97 antigen of human melanoma, carcinoma or orosomucoid-related antigen. In some embodiments, the targeting agent is configured to permit the composition to cross the blood brain barrier. In some embodiments, the targeting agent is transferrin. In some embodiments, the targeting agent is configured to permit the composition to bind with a neuron within the central nervous system. In some embodiments, the targeting agent is a synthetic tetanus toxin fragment. In some embodiments, the synthetic tetanus toxin fragment comprises an amino acid peptide fragment. In some embodiments, the amino acid peptide fragment is HLNILSTLWKYR.

In some embodiments, the ligand comprises a trigger agent. The present invention is not limited to particular type or kind of trigger agent. In some embodiments, the trigger agent is configured to have a function such as, for example, a) a delayed release of a functional group from the dendrimer, b) a constitutive release of the therapeutic agent from the dendrimer, c) a release of a functional group from the dendrimer under conditions of acidosis, d) a release of a functional group from a dendrimer under conditions of hypoxia, and e) a release of the therapeutic agent from a dendrimer in the presence of a brain enzyme. Examples of trigger agents include, but are not limited to, an ester bond, an amide bond, an ether bond, an indoquinone, a nitroheterocyle, and a nitroimidazole. In some embodiments, the trigger agent is attached with the dendrimer via a linker.

Ligands suitable for use in certain method embodiments of the present invention are not limited to a particular type or kind of imaging agent. Examples of imaging agents include, but are not limited to, fluorescein isothiocyanate (FITC), 6-TAMARA, acridine orange, and cis-parinaric acid.

Specific examples of ligands used in some method embodiments of the present invention include but are not limited to folic acid, methotrexate, camptothecin deriviatives (e.g., SN-38), and fluorescein-5(6)-carboxamidocaproic acid (FITC). In preferred embodiments, ligands include targeting agents, drugs or prodrugs, drug derivatives, and imaging agents which contain one or more carboxyl groups.

Examples of dendrimers include, but are not limited to, a polyamideamine (PAMAM) dendrimer, a polypropylamine (POPAM) dendrimer, and a PAMAM-POPAM dendrimer. In some embodiments, the dendrimer is a Baker-Huang PAMAM dendrimer (see, e.g., U.S. Provisional Patent Application Ser. No. 61/251,244; herein incorporated by reference in its entirety). The type of dendrimer used is not limited by the generation number of the dendrimer. Dendrimer molecules may be generation 0, generation 1, generation 2, generation 3, generation 4, generation 5, generation 6, generation 7, or higher than generation 7. In some embodiments, half-generation dendrimers may be used. In certain embodiments, a generation 5 amine-terminated PAMAM dendrimer is used as starting material. In some embodiments, the dendrimer is at least partially acetylated.

Dendrimers are not limited by their method of synthesis. The dendrimer may be synthesized by divergent synthesis methods or convergent synthesis methods. In certain embodiments of the present invention, dendrimer molecules may be modified. Modifications may include but are not limited to the addition of functional groups or linkers not originally present on the dendrimer. In some embodiments, all of the termini of the dendrimer molecules are modified. In some embodiments, not all of the dendrimer molecules are modified. In particularly preferred embodiments, the dendrimer comprises terminal —OH groups, without limitation to the manner in which the terminal —OH groups were introduced. Methods of introducing terminal —OH groups include but are not limited to glycidolation. In a particularly preferred embodiment, a generation 5 amine-terminated PAMAM dendrimer is modified with glycidol to result in at least one, preferably more than one, dendrimer termini bearing a 2,3-dihydroxylpropyl group. In certain embodiments, glycidolation of a generation 5 amine-terminated dendrimer occurs by reaction of the dendrimer and glycidol in a suitable solvent at room temperature, e.g. at a temperature ranging from approximately 19-27° C. In preferred embodiments, room temperature is approximately 22° C. In some embodiments, the reaction occurs in methanol. In some embodiments, the reaction occurs under an inert gas. In some embodiments, the inert gas is N₂. The glycidolation step is not limited by the duration of reaction time. Reaction time may be less than 1 hour, 1-5 hours, 5-10 hours, 10-20 hours, 20-30 hours, 30 hours or more. In preferred embodiments, at least one —OH functional group of at least one terminal 2,3-dihydroxylpropyl group serves as an attachment point for a functional ligand. In particularly preferred embodiments, at least two different functional ligands are each attached to an oxygen atom of an —OH functional group of at least one terminal 2,3-dihydroxylpropyl group, to result in the formation of an ester bond between the dendrimer and each functional ligand.

In certain embodiments, reactants are purified prior to inclusion in additional reactions, prior to analysis, and/or prior to final use. Purification methods include but are not limited to dialysis and precipitation. As non-limiting examples, purification may occur by dialysis against water, or dialysis against buffer, or dialysis against isotonic saline solution, or against any sequential combination of dialysis solutions (e.g., buffer and then water, isotonic saline solution and then water). As further non-limiting examples, purification may occur by precipitation in organic solvents such as diethyl ether, hexane, cyclohexane, ethyl acetate, acetone, chloroform, dichloromethane, tetrahydrofuran, or any combination solution of aforementioned solvents, or any combination solution of aforementioned solvents and more polar solvens such as dioxane, ethanol, methanol, N,N-dimethylformamide, dimethylsulfoxide, N,N-dimethylacetamide, 2-pyrrolidinone, and 1-methyl-2-pyrrolidinone.

In certain embodiments, the present invention provides methods for synthesizing dendrimer nanodevices, comprising: providing a dendrimer comprising terminal —OH groups; providing at least two different ligands; providing at least one ester coupling agent; and combining the dendrimer bearing terminal —OH groups, the ligands, and the ester coupling reagent under conditions such that an ester bond forms between the dendrimer and the ligands.

In certain embodiments, the invention provides methods for synthesizing functionalized dendrimer nanodevices comprising simultaneous exposure of at least two different ligands to a dendrimer comprising terminal —OH groups. In some embodiments, the exposure occurs via a one-pot synthesis reaction. In some embodiments, the dendrimer bearing terminal —OH groups comprises 2,3-dihydroxylpropyl groups. In some embodiments, the exposure is conducted in the presence of ester coupling agents.

In certain embodiments, the present invention provides methods for treating a disorder selected from the group consisting of any type of cancer or cancer-related disorder (e.g., tumor, a neoplasm, a lymphoma, or a leukemia), a neoplastic disease, osteoarthritis, rheumatoid arthritis, septic arthritis, gout and pseudo-gout, juvenile idiopathic arthritis, psoriatic arthritis, Still's disease, and ankylosing spondylitis, comprising administering to a subject suffering from the disorder a dendrimer generated with the methods of the present invention (e.g., methods for synthesizing multifunctional dendrimers (e.g., dendrimers conjugated with one or more functional groups) through, for example, initial glycidation of a dendrimer followed by simultaneous conjugation of one or more functional groups (e.g., through ester linkages in a “one pot” reaction)).

In some embodiments, the methods further involve, for example, co-administration of an agent selected from the group consisting of an antirheumatic drug, a biologicals agent, a nonsteroidal anti-inflammatory drug, an analgesic, an immunomodulator, a glucocorticoid, a TNF-a inhibitor, an IL-1 inhibitor, and a metalloprotease inhibitor. In some embodiments, the antirheumatic drug is selected from the group consisting of leflunomide, methotrexate, sulfasalazine, and hydroxychloroquine. In some embodiments, the biologicals agent is selected from the group consisting of rituximab, finfliximab, etanercept, adalimumab, and golimumab. In some embodiments, the nonsteroidal anti-inflammatory drug is selected from the group consisting of ibuprofen, celecoxib, ketoprofen, naproxen, piroxicam, and diclofenac. In some embodiments, analgesics are selected from the group consisting of acetaminophen, and tramadol. In some embodiments, the immunomodulator is selected from the group consisting of anakinra, and abatacept. In some embodiments, the glucocorticoid is selected from the group consisting of prednisone, and methylprednisone. In some embodiments, the TNF-α inhibitor is selected from the group consisting of adalimumab, certolizumab pegol, etanercept, golimumab, and infliximab. In some embodiments, the methods further involve, for example, co-administration of an anti-cancer agent, a pain relief agent, and/or a pain relief agent antagonist.

In some embodiments, the neoplastic disease includes, but is not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

In some embodiments, the disorder is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome.

In some embodiments, the disorder is a viral disease selected from the group consisting of, but not limited to, viral disease caused by hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

In some embodiments, the present invention also provides kits comprising one or more of the reagents and tools necessary to generate a dendrimer comprising one or more ligands (e.g., functional groups) wherein each ligand is attached to the dendrimer by an ester bond. Examples of such reagents include, but are not limited to, dendrimers (e.g., a polyamideamine (PAMAM) dendrimer, a polypropylamine (POPAM) dendrimer, and a PAMAM-POPAM dendrimer. In some embodiments, the dendrimer is a Baker-Huang PAMAM dendrimer) not having undergone glycidation, one or more ligands (e.g., therapeutic agents, targeting agents, trigger agents, and imaging agents), and any reagents necessary for conjugation of such ligands with such dendrimers. In some embodiments, the kit comprises a vessel designed to accommodate the one-pot dendrimer synthesis methods of the present invention (e.g., methods for synthesizing multifunctional dendrimers (e.g., dendrimers conjugated with one or more functional groups) through, for example, initial glycidation of a dendrimer followed by simultaneous conjugation of one or more functional groups (e.g., through ester linkages in a “one pot” reaction)).

Additional embodiments will be apparent to persons skilled in the relevant art based on the teachings contained herein.

### DESCRIPTION OF THE DRAWINGS

FIG. 1 shows an embodiment of methods of the present invention. The reaction depicts synthesis of G5-FA-MTX conjugate using folic acid (FA), methotrexate (MTX) and G5 polyamidoamine (PAMAM) dendrimer. Reagents and conditions: (a) glycidol, methanol, room temperature, 24 hours; (b) FA, MTX, 2-chloro-1-methylpyridinium iodide, 4-(dimethylamino)pyridine, dimethylsulfoxide, room temperature, 24 hours.

FIG. 2 shows a comparison of ¹HNMR spectra of G5 PAMAM dendrimer, hydroxyl-terminated G5 PAMAM dendrimer, and conjugate of G5 PAMAM dendrimer, FA, and MTX.

FIG. 3 shows an ¹H NMR spectrum of conjugate of G5 PAMAM dendrimer, FA, and MTX.

FIG. 4 shows an enlarged scale of ¹H NMR spectrum of proton-7 of conjugated FA and MTX (bottom panel). The positions of proton-7 of FA and MTX, respectively, are shown in the top panel.

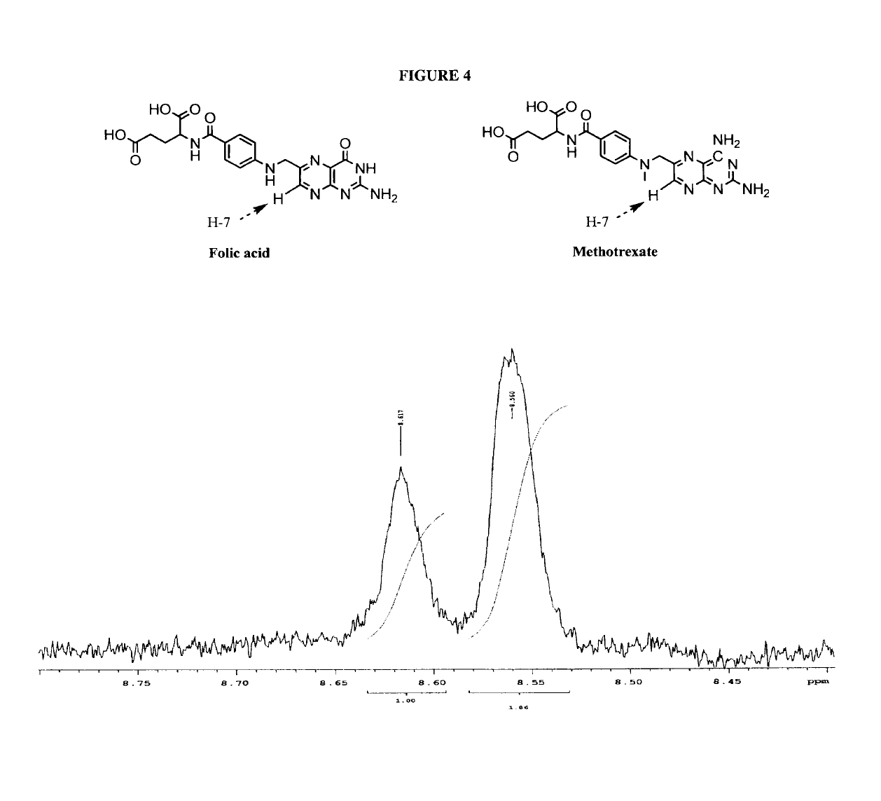

FIG. 5 shows MALDI-TOF mass spectra of G5 PAMAM dendrimer, hydroxyl-terminated G5 PAMAM dendrimer, and conjugate of G5 PAMAM dendrimer, folic acid, and methotrexate.

FIG. 6 shows an HPLC chromatogram of conjugate (G5-FA-MTX) under UV 280 nm.

FIG. 7 shows an HPLC chromatogram of hydroxyl-terminated G5 PAMAM dendrimer under UV 210 nm.

FIG. 8 shows an HPLC chromatogram of conjugate (G5-FA-MTX) under UV 210 nm.

FIG. 9 shows a graph showing the differential molar mass fractions versus molar mass for G5 PAMAM dendrimer, hydroxyl-terminated G5 PAMAM dendrimer, and conjugate of G5 PAMAM dendrimer, FA and MTX.

FIG. 10 shows competition of G5-FA-MTX synthesized using the one-pot approach with G5-FI-FA-MTX. KB cells were treated with the two conjugates simultaneously, and the mean FL1 fluorescence of 10,000 cells was determined by flow cytometry. The data is expressed as the percent fluorescence obtained for the binding of 100 nM G5-FI-FA-MTX in the absence of G5-FA-MTX. As indicated by the arrow, the concentration of G5-FA-MTX that is required for 50% reduction in binding of the G5-FI-FA-MTX is ˜60 nM.

FIG. 11 shows in vitro cytotoxicity of the newly synthesized G5-FA-MTX. KB cells were treated with different concentrations of the newly synthesized G5-FA-MTX (triangle symbols), and free MTX (circle symbols). The data also shows the cytotoxicity of G5-FA-MTX synthesized through the classic synthetic pathway in which the FA and MTX were conjugated through amide and ester linkages, respectively (square symbols). The cells were treated with the drugs for 5 days, with one change of fresh medium/drug after 3 days, and the live cells were quantified by the XTT assay. The data represents the mean±SE of 5 replicate cell samples in a representative experiment, with identical data obtained in 3 independent experiments.

FIG. 12 shows results of an experiment in which assessment of cytotoxicity was performed using the XTT reagent, which was converted to a fluorescent product by the viable cells in each well (of a 96-well plate). All the “one-pot” synthesized G5-MTX and G5-FA-MTX conjugates showed dose-dependent cytotoxicity in KB cells. Notably, G5-MTX-FL, without having the traditional targeting ligand FA, also displayed high cytotoxicity. These data demonstrate, for example, that the polyvalent MTX conjugate by itself can internalize into the tumor cells and cause cytotoxicity.

FIG. 13 shows an embodiment of the methods of the present invention. The reaction depicts synthesis of G5 dendrimer-FA-MTX-FTIC.

FIG. 14 shows an embodiment of methods of the present invention. The reaction depicts synthesis of G5 dendrimer-FA-7-Ethyl-10-Hydroxycamptothecin (SN-38).

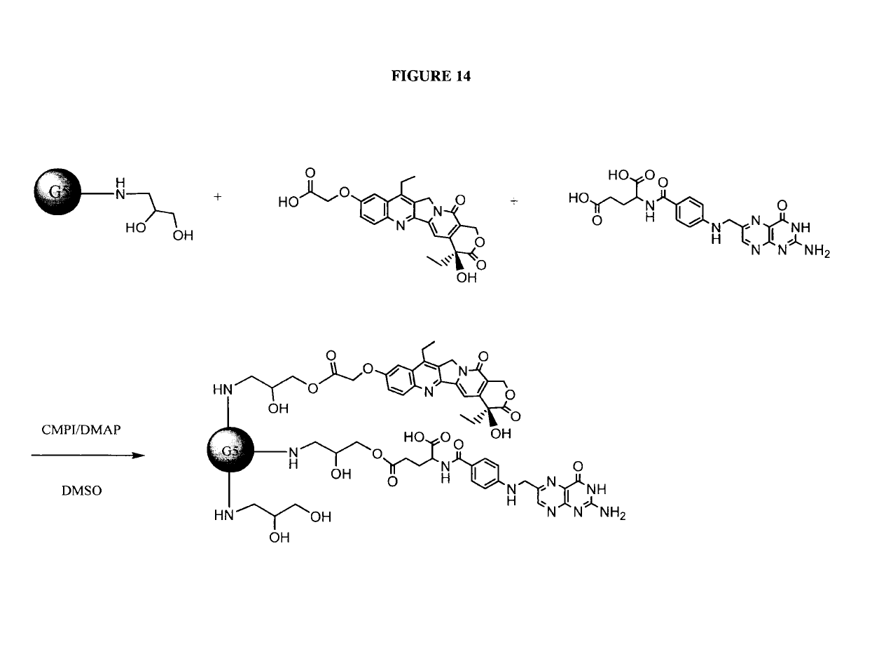

FIG. 15 shows the uptake pattern of targeted conjugates G5-FA-MTX-FL and G5-FA-FL by KB cells as analyzed by flow cytometry in a 24-well plate. Both G5-FA-MTX-FL and G5-FA-FL displayed high uptake rates. The addition of 50-fold excess free FA successfully blocked the uptake of these conjugates (dashed lines), indicating the specificity of the internalization. Furthermore, G5-MTX-FL exhibited a lowered but relatively significant uptake pattern. The uptake of G5-MTX-FL was also inhibited by excess free FA. These data demonstrate, for example, that MTX can target the folic acid receptor for internalization.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. A “preliminary diagnosis” is one based only on visual (e.g., CT scan or the presence of a lump) and antigen tests (e.g., PSMA).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “drug” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “purified” or “to purify” or “compositional purity” refers to the removal of components (e.g., contaminants) from a sample or the level of components (e.g., contaminants) within a sample. For example, unreacted moieties, degradation products, excess reactants, or byproducts are removed from a sample following a synthesis reaction or preparative method.

As used herein, the term “nanodevice” or “nanodevices” refer, generally, to compositions comprising dendrimers of the present invention. As such, a nanodevice may refer to a composition comprising a dendrimer and metal nanoparticles (e.g., iron oxide nanoparticles (e.g., poly(styrene sulfonate) (PSS)-coated iron oxide nanoparticles)) of the present invention that may contain one or more functional groups (e.g., a therapeutic agent) conjugated to the dendrimer. A nanodevice may also refer to a composition comprising two or more different dendrimers of the present invention.

As used herein, the term “degradable linkage,” when used in reference to a polymer (e.g., PEG-hRNase conjugate of the present invention), refers to a conjugate that comprises a physiologically cleavable linkage (e.g., a linkage that can be hydrolyzed (e.g., in vivo) or otherwise reversed (e.g., via enzymatic cleavage). Such physiologically cleavable linkages include, but are not limited to, ester, carbonate ester, carbamate, sulfate, phosphate, acyloxyalkyl ether, acetal, and ketal linkages (See, e.g., U.S. Pat. No. 6,838,076, herein incorporated by reference in its entirety). Similarly, the conjugate may comprise a cleavable linkage present in the linkage between the polymer and hRNase, or, may comprise a cleavable linkage present in the polymer itself (e.g., such that when cleaved, a small portion of the polymer remains on the hRNase molecule) (See, e.g., U.S. Pat. App. Nos. 20050158273 and 20050181449, each of which is herein incorporated by reference in its entirety). For example, a PEG polymer comprising an ester linkage can be utilized for conjugation to hRNase to create a PEG-hRNase conjugate (See, e.g., Kuzlowski et al., Biodrugs, 15, 419-429 (2001). A conjugate that comprises a degradable linkage of the present invention is capable of generating hRNase that is free (e.g., completely or partially free) of the polymer (e.g., in vivo after hydrolysis of the linkage).

A “physiologically cleavable” or “hydrolysable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes.

A “hydrolytically stable” linkage or bond refers to a chemical bond (e.g., typically a covalent bond) that is substantially stable in water (i.e., does not undergo hydrolysis under physiological conditions to any appreciable extent over an extended period of time). Examples of hydrolytically stable linkages include, but are not limited to, carbon-carbon bonds (e.g., in aliphatic chains), ethers, amides, urethanes, and the like.

As used herein, the term “NAALADase inhibitor” refers to any one of a multitude of inhibitors for the neuropeptidase NAALADase (N-acetylated-alpha linked acidic dipeptidase). Such inhibitors of NAALADase have been well characterizied. For example, an inhibitor can be selected from the group comprising, but not limited to, those found in U.S. Pat. No. 6,011,021, herein incorporated by reference in its entirety.

As used herein, the term “click chemistry” refers to chemistry tailored to generate substances quickly and reliably by joining small modular units together (see, e.g., Kolb et al. (2001) Angewandte Chemie Intl. Ed. 40:2004-2011; Evans (2007) Australian J. Chem. 60:384-395; Carlmark et al. (2009) Chem. Soc. Rev. 38:352-362; each herein incorporated by reference in its entirety).

As used herein, the term “ligand” refers to any moiety covalently attached (e.g., conjugated) to a dendrimer branch; in preferred embodiments, such conjugation is indirect (e.g., an intervening moiety exists between the dendrimer branch and the ligand) rather than direct (e.g., no intervening moiety exists between the dendrimer branch and the ligand). Indirect attachment of a ligand to a dendrimer may exist where a scaffold compound (e.g., triazine scaffold) intervenes. In preferred embodiments, ligands have functional utility for specific applications, e.g., for therapeutic, targeting, imaging, or drug delivery function(s). The terms “ligand”, “conjugate”, and “functional group” may be used interchangeably.

As used herein, an “ester coupling agent” refers to a reagent that can facilitate the formation of an ester bond between two reactants. The present invention is not limited to any particular coupling agent or agents. Examples of coupling agents include but are not limited to 2-chloro-1-methylpyridinium iodide and 4-(dimethylamino)pyridine, or dicyclohexylcarbodiimide and 4-(dimethylamino) pyridine or diethyl azodicarboxylate and triphenylphosphine or other carbodiimide coupling agent and 4-(dimethylamino)pyridine.

As used herein, the term “glycidolate” refers to the addition of a 2,3-dihydroxylpropyl group to a reagent using glycidol as a reactant. In some embodiments, the reagent to which the 2,3-dihydroxylpropyl groups are added is a dendrimer. In some embodiments, the dendrimer is a PAMAM dendrimer. Glycidolation may be used generally to add terminal hydroxyl functional groups to a reagent.

As used herein, the term “amino alcohol” or “amino-alcohol” refers to any organic compound containing both an amino and an aliphatic hydroxyl functional group (e.g., which may be an aliphatic or branched aliphatic or alicyclic or hetero-alicyclic compound containing an amino group and one or more hydroxyl(s)). The generic structure of an amino alcohol may be expressed as NH₂—R-(OH)ₘ wherein m is an integer, and wherein R comprises at least two carbon molecules (e.g., at least 2 carbon molecules, 10 carbon molecules, 25 carbon molecules, 50 carbon molecules).

As used herein, the term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, conjugation between a dendrimer (e.g., a terminal arm of a dendrimer) and a functional ligand is accomplished during a “one-pot” reaction. The term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, a one-pot reaction occurs wherein a hydroxyl-terminated dendrimer (e.g., HO-PAMAM dendrimer) is reacted with one or more functional ligands (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino)pyridine) (see, e.g., U.S. Patent App. No. 61/226,993, herein incorporated by reference in its entirety).

As used herein, the term “solvent” refers to a medium in which a reaction is conducted. Solvents may be liquid but are not limited to liquid form. Solvent categories include but are not limited to nonpolar, polar, protic, and aprotic.

As used herein, the term “dialysis” refers to a purification method in which the solution surrounding a substance is exchanged over time with another solution. Dialysis is generally performed in liquid phase by placing a sample in a chamber, tubing, or other device with a selectively permeable membrane. In some embodiments, the selectively permeable membrane is cellulose membrane. In some embodiments, dialysis is performed for the purpose of buffer exchange. In some embodiments, dialysis may achieve concentration of the original sample volume. In some embodiments, dialysis may achieve dilution of the original sample volume.

As used herein, the term “precipitation” refers to purification of a substance by causing it to take solid form, usually within a liquid context. Precipitation may then allow collection of the purified substance by physical handling, e.g. centrifugation or filtration.

As used herein, the term “Baker-Huang dendrimer” or “Baker-Huang PAMAM dendrimer” refers to a dendrimer comprised of branching units of structure:

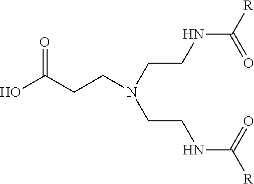

wherein R comprises a carbon-containing functional group (e.g., CF₃). In some embodiments, the branching unit is activated to its HNS ester. In some embodiments, such activation is achieved using TSTU. In some embodiments, EDA is added. In some embodiments, the dendrimer is further treated to replace, e.g., CF₃ functional groups with NH₂ functional groups; for example, in some embodiments, a CF₃-containing version of the dendrimer is treated with K₂CO₃ to yield a dendrimer with terminal NH₂ groups (for example, as shown in U.S. patent application Ser. No. 12/645,081, herein incorporated by reference in its entirety). In some embodiments, terminal groups of a Baker-Huang dendrimer are further derivatized and/or further conjugated with other moieties. For example, one or more functional ligands (e.g., for therapeutic, targeting, imaging, or drug delivery function(s)) may be conjugated to a Baker-Huang dendrimer, either via direct conjugation to terminal branches or indirectly (e.g., through linkers, through other functional groups (e.g., through an OH— functional group)). In some embodiments, the order of iterative repeats from core to surface is amide bonds first, followed by tertiary amines, with ethylene groups intervening between the amide bond and tertiary amines In preferred embodiments, a Baker-Huang dendrimer is synthesized by convergent synthesis methods.

### DETAILED DESCRIPTION OF THE INVENTION

Conjugation of ligands (e.g., functional groups) with dendrimers is typically accomplished through amide and ester linkages, respectively, using a multiple step synthetic route (Kukowska-Latallo et al. (2005) Cancer Res. 65:5317-5324; Quintana et al. (2002) Pharmaceutical Res. 19:1310-1316; Majoros et al. (2005) J. Med. Chem. 48:5892-5899; each herein incorporated by reference in its entirety). The synthetic steps involved partial acetylation of the dendrimer, conjugation of the functional agent using EDC chemistry through amide bonds, glycidation of the remaining amino groups, and finally conjugation of MTX through ester linkage through some of the glycidol moieties. Variability in efficiency of each of these synthetic steps resulted in batch-to-batch reproducibility issues which limited the application of this technology.

The present invention overcomes such synthetic limitations through providing simiplified methods for synthesizing conjugated dendrimers. In particular, the present invention provides methods for synthesizing multifunctional dendrimers (e.g., dendrimers conjugated with one or more functional groups) through, for example, initial glycidation of a dendrimer followed by simultaneous conjugation of one or more functional groups (e.g., through ester linkages in a “one pot” reaction). The novel methods of the present invention represent a significant improvement over previous synthetic methods in terms of, for example, lower total reaction time, higher yield, and greater ease of manufacturing. In addition, in one non-limiting example, a dendrimer-FA-MTX (PAMAM dendrimer/folic acid/methotrexate) conjugate synthesized by the novel methods of the present invention displayed similar cytotoxic potency as compared to dendrimer-FA-MTX conjugates that had been synthesized using alternative synthetic approaches. The methods are not limited by the nature of the ligand, the nature of the dendrimer, or the nature of the one-pot synthesis reaction.

The synthesis methods of certain embodiments of the present invention are reproducible and feasible for large scale synthesis. In one non-limiting example, the molecules of FA and MTX attached to each dendrimer molecule were easily adjusted to generate conjugates with a variety of desired ratios of the two ligands. The final conjugate and all the intermediate products were characterized by ¹H NMR, MALDI-TOF, GPC, and HPLC to confirm the efficiency of the synthetic steps. Indeed, the present invention provides analytical approaches for calculating molecules of functional groups (e.g., folic acid and methotrexate) attached to a particular dendrimer molecule through, for example, combining characterization techniques of ¹H NMR and MALDI-TOF.

Accordingly, in certain embodiments, the present invention provides methods for synthesizing dendrimer conjugates (e.g., dendrimers conjugated with one or more functional groups) through, for example, initial glycidolation of a dendrimer molecule (e.g., such that the terminal dendrimer molecule is rendered with terminal hydroxyl groups instead of terminal NH₂ groups), and conjugation of one or more functional groups (e.g., therapeutic agents, targeting agents, trigger agents, and imaging agents) with the glycidolated dendrimer molecule.

The methods are not limited to a particular manner of dendrimer glycidation. In some embodiments, dendrimer glycidation involves exposure and mixing of dendrimer molecules with glycidol.

The methods are not limited to a particular manner of conjugating the functional groups with the glycidated dendrimer molecule. In some embodiments, the conjugation involves ester linkage between a terminal hydroxyl group on the glycidated dendrimer and the functional group. In some embodiments, the conjugation of the one or more functional groups occurs simultaneously (e.g., two or more different functional groups are simultaneously exposed to the glycidolated dendrimer). In some embodiments, the conjugation occurs via a one-pot synthesis reaction. As noted, methods for synthesizing dendrimer conjugates through such techniques (e.g., initial glycidolation of a dendrimer followed by simultaneous conjugation of one or more functional groups (e.g., through ester linkages in a one-pot reaction)) results in, in comparison to previous synthetic methods, lower total reaction time, higher dendrimer conjugate yield, and greater ease of manufacturing.

The present invention is not limited to a particular one-pot synthesis technique. In certain embodiments, the one-pot synthesis reaction occurs by combining all reactants in a single reaction vessel. Reactants may be added simultaneously or sequentially. The method is not limited by the order of addition of reactants, nor by the amount of time passing between any sequential addition of reactants. The reaction is not limited by the relative proportion of reactants. In certain embodiments, it is possible to tune the reaction by altering the relative proportion of reactions. In one non-limiting example, the reaction feed molar ratio of different ligands may be altered to affect the average number and relative proportion of ligands attached to the dendrimer. In certain embodiments where two different ligands are attached to a dendrimer (e.g. a glycidolated G5 PAMAM dendrimer), where the reaction feed molar ratio is represented as A:B:C where A is the relative molarity of ligand 1, B is the relative molarity of ligand 2, and C is the relative molarity of glycidolated G5 PAMAM dendrimer, the value of each of A, B, and C may be varied from 1 to 100. In preferred embodiments, the value of C is held at 1 and the values of each of A and B range from 1 to 50.

The one-pot synthesis reaction method is not limited by the size or shape of the vessel in which it is performed or the material from which the vessel is made. The reaction is not limited by reaction volume. Volume of the reaction may be less than 5 ml, 5-10 ml, 10-20 ml, 20-50 ml, 50-100 ml, 100-1000 ml, 1 L-25 L, 25-50 L, 50 L or more. The reaction is not limited by the pressure at which it is performed. In preferred embodiments, the reaction is conducted at atmospheric pressure. In some embodiments, the reaction occurs under an inert gas. In preferred embodiments, the inert gas is N₂. The reaction is not limited by the temperature at which it is conducted. In preferred embodiments, the reaction is performed at room temperature, e.g. at a temperature ranging from approximately 19-27° C. In particularly preferred embodiments, room temperature is approximately 22° C. The reaction is not limited by the duration of reaction time. Reaction time may be less than 1 hour, 1-5 hours, 5-10 hours, 10-20 hours, 20-30 hours, 30 hours or more. In preferred embodiments, the reaction occurs for 24 hours. In preferred embodiments, the reaction occurs in a suitable solvent system. Suitable solvent systems include but are not limited to polar solvent systems. Examples of polar solvent systems include but are not limited to dimethylsulfoxide (DMSO); N,N-dimethylformamide; N-N-dimethylacetamide; 2-pyrrolidinone; 1-methyl-2-pyrrolidinone; dioxane or any combination thereof.

In certain embodiments, the formation of ester bonds (e.g., between a functional group and a terminal hydroxyl group on a glycidolated dendrimer) is facilitated by the presence of ester coupling agents. Examples of ester coupling agents include but are not limited to 2-chloro-1-methylpyridinium iodide and 4-(dimethylamino)pyridine, or dicyclohexylcarbodiimide and 4-(dimethylamino)pyridine or diethyl azodicarboxylate and triphenylphosphine or other carbodiimide coupling agent and 4-(dimethylamino)pyridine.

Accordingly, the present invention relates to novel methods of synthesis of therapeutic and diagnostic dendrimers. In particular, the present invention is directed to novel dendrimer conjugates, methods of synthesizing the same, compositions comprising the conjugates, as well as systems and methods utilizing the conjugates (e.g., in diagnostic and/or therapeutic settings (e.g., for the delivery of therapeutics, imaging, and/or targeting agents (e.g., in disease (e.g., cancer) diagnosis and/or therapy, pain therapy, etc.)). Accordingly, dendrimer conjugates of the present invention may further comprise at least two different components for targeting, imaging, sensing, and/or providing a therapeutic or diagnostic material and/or monitoring response to therapy. Furthermore, the novel synthesis methods of certain embodiments of the present invention provide significant advantages with regard to total reaction time and simplicity.

The present invention is not limited to the use of particular types and/or kinds of dendrimers (e.g., a dendrimer conjugated with at least one functional group). Indeed, dendrimeric polymers have been described extensively (See, e.g., Tomalia, Advanced Materials 6:529 (1994); Angew, Chem. Int. Ed. Engl., 29:138 (1990); incorporated herein by reference in their entireties). Dendrimer polymers are synthesized as defined spherical structures typically ranging from 1 to 20 nanometers in diameter. Methods for manufacturing a G5 PAMAM dendrimer with a protected core are known (U.S. patent application Ser. No. 12/403,179; herein incorporated by reference in its entirety). In preferred embodiments, the protected core diamine is NH₂—CH₂—CH₂—NHPG. Molecular weight and the number of terminal groups increase exponentially as a function of generation (the number of layers) of the polymer. In some embodiments of the present invention, half generation PAMAM dendrimers are used. For example, when an ethylenediamine (EDA) core is used for dendrimer synthesis, alkylation of this core through Michael addition results in a half-generation molecule with ester terminal groups; amidation of such ester groups with excess EDA results in creation of a full-generation, amine-terminated dendrimer (Majoros et al., Eds. (2008) Dendrimer-based Nanomedicine, Pan Stanford Publishing Pte. Ltd., Singapore, p. 42). Different types of dendrimers can be synthesized based on the core structure that initiates the polymerization process. In some embodiments, the PAMAM dendrimers are “Baker-Huang dendrimers” or “Baker-Huang PAMAM dendrimers” (see, e.g., U.S. Provisional Patent Application Ser. No. 61/251,244; herein incorporated by reference in its entirety).

The dendrimer core structures dictate several characteristics of the molecule such as the overall shape, density and surface functionality (See, e.g., Tomalia et al., Chem. Int. Ed. Engl., 29:5305 (1990)). Spherical dendrimers can have ammonia as a trivalent initiator core or ethylenediamine (EDA) as a tetravalent initiator core. Rod-shaped dendrimers (See, e.g., Yin et al., J. Am. Chem. Soc., 120:2678 (1998)) use polyethyleneimine linear cores of varying lengths; the longer the core, the longer the rod. Dendritic macromolecules are available commercially in kilogram quantities and are produced under current good manufacturing processes (GMP) for biotechnology applications.

Dendrimers may be characterized by a number of techniques including, but not limited to, electrospray-ionization mass spectroscopy, ¹³C nuclear magnetic resonance spectroscopy, ¹H nuclear magnetic resonance spectroscopy, size exclusion chromatography with multi-angle laser light scattering, ultraviolet spectrophotometry, capillary electrophoresis and gel electrophoresis. These tests assure the uniformity of the polymer population and are important for monitoring quality control of dendrimer manufacture for GMP applications and in vivo usage.

Numerous U.S. patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describe methods of making dense star polymers with terminal densities greater than conventional star polymers. These polymers have greater/more uniform reactivity than conventional star polymers, i.e. 3rd generation dense star polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

U.S. Pat. No. 6,471,968 describes a dendrimer complex comprising covalently linked first and second dendrimers, with the first dendrimer comprising a first agent and the second dendrimer comprising a second agent, wherein the first dendrimer is different from the second dendrimer, and where the first agent is different than the second agent.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

PAMAM dendrimers are highly branched, narrowly dispersed synthetic macromolecules with well-defined chemical structures. PAMAM dendrimers can be easily modified and conjugated with multiple functionalities such as targeting molecules, imaging agents, and drugs (Thomas et al. (2007) Poly(amidoamine) Dendrimer-based Multifunctional Nanoparticles, in Nanobiotechnology: Concepts, Methods and Perspectives, Merkin, Ed., Wiley-VCH; herein incorporated by reference in its entirety). They are water soluble, biocompatible, and cleared from the blood through the kidneys (Peer et al. (2007) Nat. Nanotechnol. 2:751-760; herein incorporated by reference in its entirety) which eliminates the need for biodegradability. Because of these desirable properties, PAMAM dendrimers have been widely investigated for drug delivery (Esfand et al. (2001) Drug Discov. Today 6:427-436; Patri et al. (2002) Curr. Opin. Chem. Biol. 6:466-471; Kukowska-Latallo et al. (2005) Cancer Res. 65:5317-5324; Quintana et al. (2002) Pharmaceutical Res. 19:1310-1316; Thomas et al. (2005) J. Med. Chem. 48:3729-3735; each herein incorporated by reference in its entirety), gene therapy (KukowskaLatallo et al. (1996) PNAS 93:4897-4902; Eichman et al. (2000) Pharm. Sci. Technolo. Today 3:232-245; Luo et al. (2002) Macromol. 35:3456-3462; each herein incorporated by reference in its entirety), and imaging applications (Kobayashi et al. (2003) Bioconj. Chem. 14:388-394; herein incorporated by reference in its entirety).

The use of dendrimers as metal ion carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjuvants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polyeation non-covalently coupled to the polynucleotide.

Dendrimer-antibody conjugates for use in in vitro diagnostic applications have previously been demonstrated (See, e.g., Singh et al., Clin. Chem., 40:1845 (1994)), for the production of dendrimer-chelant-antibody constructs, and for the development of boronated dendrimer-antibody conjugates (for neutron capture therapy); each of these latter compounds may be used as a cancer therapeutic (See, e.g., Wu et al., Bioorg. Med. Chem. Lett., 4:449 (1994); Wiener et al., Magn. Reson. Med. 31:1 (1994); Barth et al., Bioconjugate Chem. 5:58 (1994); and Barth et al.).

Some of these conjugates have also been employed in the magnetic resonance imaging of tumors (See, e.g., Wu et al., (1994) and Wiener et al., (1994), supra). Results from this work have documented that, when administered in vivo, antibodies can direct dendrimer-associated therapeutic agents to antigen-bearing tumors. Dendrimers also have been shown to specifically enter cells and carry either chemotherapeutic agents or genetic therapeutics. In particular, studies show that cisplatin encapsulated in dendrimer polymers has increased efficacy and is less toxic than cisplatin delivered by other means (See, e.g., Duncan and Malik, Control Rel. Bioact. Mater. 23:105 (1996)).

Dendrimers have also been conjugated to fluorochromes or molecular beacons and shown to enter cells. They can then be detected within the cell in a manner compatible with sensing apparatus for evaluation of physiologic changes within cells (See, e.g., Baker et al., Anal. Chem. 69:990 (1997)). Finally, dendrimers have been constructed as differentiated block copolymers where the outer portions of the molecule may be digested with either enzyme or light-induced catalysis (See, e.g., Urdea and Hom, Science 261:534 (1993)). This allows the controlled degradation of the polymer to release therapeutics at the disease site and provides a mechanism for an external trigger to release the therapeutic agents.

The present invention is not limited to the use of particular therapeutic agents. In some embodiments, the therapeutic agents are effective in treating autoimmune disorders and/or inflammatory disorders (e.g., arthritis). Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone), TNF-a inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, parenteral gold or oral gold.

In some embodiments, the therapeutic agents are effective in treating cancer (see, e.g., U.S. Pat. Nos. 6,471,968, 7,078,461, and U.S. patent application Ser. Nos. 09/940,243, 10/431,682, 11/503,742, 11/661,465, 11/523,509, 12/403,179, 12/106,876, and 11/827,637; and U.S. Provisional Patent Application Ser. Nos. 61/256,759, 61/140,840, 61/091,608, 61/097,780, 61/101,461, 61/237,172, 61/229,168, 61/221,596, and 61/251,244; each herein incorporated by reference in their entireties).

In some embodiments, the therapeutic agent is conjugated to a trigger agent. The present invention is not limited to particular types or kinds of trigger agents.

In some embodiments, sustained release (e.g., slow release over a period of 24-48 hours) of the therapeutic agent is accomplished through conjugating the therapeutic agent (e.g., directly) (e.g., indirectly through one or more additional functional groups) to a trigger agent that slowly degrades in a biological system (e.g., amide linkage, ester linkage, ether linkage). In some embodiments, constitutively active release of the therapeutic agent is accomplished through conjugating the therapeutic agent to a trigger agent that renders the therapeutic agent constitutively active in a biological system (e.g., amide linkage, ether linkage).

In some embodiments, release of the therapeutic agent under specific conditions is accomplished through conjugating the therapeutic agent (e.g., directly) (e.g., indirectly through one or more additional functional groups) to a trigger agent that degrades under such specific conditions (e.g., through activation of a trigger molecule under specific conditions that leads to release of the therapeutic agent). For example, once a conjugate (e.g., a therapeutic agent conjugated with a trigger agent and a targeting agent) arrives at a target site in a subject (e.g., a tumor, or a site of inflammation), components in the target site (e.g., a tumor associated factor, or an inflammatory or pain associated factor) interact with the trigger agent thereby initiating cleavage of the therapeutic agent from the trigger agent. In some embodiments, the trigger agent is configured to degrade (e.g., release the therapeutic agent) upon exposure to a tumor-associated factor (e.g., hypoxia and pH, an enzyme (e.g., glucuronidase and/or plasmin), a cathepsin, a matrix metalloproteinase, a hormone receptor (e.g., integrin receptor, hyaluronic acid receptor, luteinizing hormone-releasing hormone receptor, etc.), cancer and/or tumor specific DNA sequence), an inflammatory associated factor (e.g., chemokine, cytokine, etc.) or other moiety.

In some embodiments, the present invention provides a therapeutic agent conjugated with a trigger agent that is sensitive to (e.g., is cleaved by) hypoxia (e.g., indolequinone). Hypoxia is a feature of several disease states, including cancer, inflammation and rheumatoid arthritis, as well as an indicator of respiratory depression (e.g., resulting from analgesic drugs).

Advances in the chemistry of bioreductive drug activation have led to the design of various hypoxia-selective drug delivery systems in which the pharmacophores of drugs are masked by reductively cleaved groups. In some embodiments, the trigger agent is utilizes a quinone, N-oxide and/or (hetero)aromatic nitro groups. For example, a quinone present in a conjugate is reduced to phenol under hypoxia conditions, with spontaneous formation of lactone that serves as a driving force for drug release. In some embodiments, a heteroaromatic nitro compound present in a conjugate (e.g., a therapeutic agent conjugated (e.g., directly or indirectly) with a trigger agent) is reduced to either an amine or a hydroxylamine, thereby triggering the spontaneous release of a therapeutic agent. In some embodiments, the trigger agent degrades upon detection of reduced pO₂ concentrations (e.g., through use of a redox linker).

The concept of pro-drug systems in which the pharmacophores of drugs are masked by reductively cleavable groups has been widely explored by many research groups and pharmaceutical companies (see, e.g., Beall, H. D., et al., Journal of Medicinal Chemistry, 1998. 41(24): p. 4755-4766; Ferrer, S., D. P. Naughton, and M. D. Threadgill, Tetrahedron, 2003. 59(19): p. 3445-3454; Naylor, M. A., et al., Journal of Medicinal Chemistry, 1997. 40(15): p. 2335-2346; Phillips, R. M., et al., Journal of Medicinal Chemistry, 1999. 42(20): p. 4071-4080; Zhang, Z., et al., Organic & Biomolecular Chemistry, 2005. 3(10): p. 1905-1910; each of which are herein incorporated by reference in their entireties). Several such hypoxia activated pro-drugs have been advanced to clinical investigations, and work in relevant oxygen concentrations to prevent cerebral damage. The present invention is not limited to particular hypoxia-activated trigger agents. In some embodiments, the hypoxia-activated trigger agents include, but are not limited to, indolequinones, nitroimidazoles, and nitroheterocycles (see, e.g., Damen, E. W. P., et al., Bioorganic & Medicinal Chemistry, 2002. 10(1): p. 71-77; Hay, M. P., et al., Journal of Medicinal Chemistry, 2003. 46(25): p. 5533-5545; Hay, M. P., et al., Journal of the Chemical Society-Perkin Transactions 1, 1999(19): p. 2759-2770; each herein incorporated by reference in their entireties).

In some embodiments, the trigger agent is sensitive to (e.g., is cleaved by) and/or associates with a tumor-associated enzyme. For example, in some embodiments, the trigger agent that is sensitive to (e.g., is cleaved by) and/or associates with a glucuronidase. Glucuronic acid can be attached to several anticancer drugs via various linkers. These anticancer drugs include, but are not limited to, doxorubicin, paclitaxel, docetaxel, 5-fluorouracil, 9-aminocamtothecin, as well as other drugs under development. These pro-drugs are generally stable at physiological pH and are significantly less toxic than the parent drugs.

In some embodiments, the trigger agent is sensitive to (e.g., is cleaved by) and/or associates with brain enzymes. For example, trigger agents such as indolequinone are reduced by brain enzymes such as, for example, diaphorase (DT-diaphorase) (see, e.g., Damen, E. W. P., et al., Bioorganic & Medicinal Chemistry, 2002. 10(1): p. 71-77; herein incorporated by reference in its entirety). For example, in such embodiments, the antagonist is only active when released during hypoxia to prevent respiratory failure.

In some embodiments, the trigger agent is sensitive to (e.g., is cleaved by) and/or associates with a protease. The present invention is not limited to any particular protease. In some embodiments, the protease is a cathepsin. In some embodiments, a trigger comprises a Lys-Phe-PABC moiety (e.g., that acts as a trigger). In some embodiments, a Lys-Phe-PABC moiety linked to doxorubicin, mitomycin C, and paclitaxel are utilized as a trigger-therapeutic conjugate in a conjugated dendrimer provided herein (e.g., a dendrimer conjugated with one or more functional groups through, for example, initial glycidation of the dendrimer followed by simultaneous conjugation of one or more functional groups (e.g., through ester linkages in a “one pot” reaction)) (e.g., that serve as substrates for lysosomal cathepsin B or other proteases expressed (e.g., overexpressed) in tumor cells). In some embodiments, utilization of a 1,6-elimination spacer/linker is utilized (e.g., to permit release of therapeutic drug post activation of trigger).

In some embodiments, the trigger agent is sensitive to (e.g., is cleaved by) and/or associates with plasmin. The serine protease plasmin is over expressed in many human tumor tissues. Tripeptide specifiers (e.g., including, but not limited to, Val-Leu-Lys) have been identified and linked to anticancer drugs through elimination or cyclization linkers.

In some embodiments, the trigger agent is sensitive to (e.g., is cleaved by) and/or associates with a matrix metalloprotease (MMP). In some embodiments, the trigger agent is sensitive to (e.g., is cleaved by) and/or that associates with β-Lactamase (e.g., a β-Lactamase activated cephalosporin-based pro-drug).

In some embodiments, the trigger agent is sensitive to (e.g., is cleaved by) and/or activated by a receptor (e.g., expressed on a target cell (e.g., a tumor cell)).

In some embodiments, the trigger agent that is sensitive to (e.g., is cleaved by) and/or activated by a nucleic acid. Nucleic acid triggered catalytic drug release can be utilized in the design of chemotherapeutic agents. Thus, in some embodiments, disease specific nucleic acid sequence is utilized as a drug releasing enzyme-like catalyst (e.g., via complex formation with a complimentary catalyst-bearing nucleic acid and/or analog). In some embodiments, the release of a therapeutic agent is facilitated by the therapeutic component being attached to a labile protecting group, such as, for example, cisplatin or methotrexate being attached to a photolabile protecting group that becomes released by laser light directed at cells emitting a color of fluorescence (e.g., in addition to and/or in place of target activated activation of a trigger component of a conjugated dendrimer of the present invention (e.g., a dendrimer conjugated with one or more functional groups through, for example, initial glycidation of the dendrimer followed by simultaneous conjugation of one or more functional groups (e.g., through ester linkages in a “one pot” reaction)). In some embodiments, the therapeutic device also may have a component to monitor the response of the tumor to therapy. For example, where a therapeutic agent of the dendrimer induces apoptosis of a target cell (e.g., a cancer cell (e.g., a prostate cancer cell)), the caspase activity of the cells may be used to activate a green fluorescence. This allows apoptotic cells to turn orange, (combination of red and green) while residual cells remain red. Any normal cells that are induced to undergo apoptosis in collateral damage fluoresce green.

In some embodiments, therapeutic agent is conjugated (e.g., directly or indirectly) to a targeting agent. The present invention is not limited to any particular targeting agent. In some embodiments, targeting agents are conjugated to the therapeutic agents for delivery of the therapeutic agents to desired body regions (e.g., to the central nervous system (CNS); to a tissue region associated with an inflammatory disorder and/or an autoimmune disorder (e.g., arthritis)). The targeting agents are not limited to targeting specific body regions.

In some embodiments, the targeting agent is a moiety that has affinity for a tumor associated factor. For example, a number of targeting agents are contemplated to be useful in the present invention including, but not limited to, RGD sequences, low-density lipoprotein sequences, a NAALADase inhibitor, epidermal growth factor, and other agents that bind with specificity to a target cell (e.g., a cancer cell)).

The present invention is not limited to cancer and/or tumor targeting agents. Indeed, conjugated dendrimers of the present invention (e.g., a dendrimer conjugated with one or more functional groups through, for example, initial glycidation of the dendrimer followed by simultaneous conjugation of one or more functional groups (e.g., through ester linkages in a “one pot” reaction)) can be targeted (e.g., via a linker conjugated to the dendrimer wherein the linker comprises a targeting agent) to a variety of target cells or tissues (e.g., to a biologically relevant environment) via conjugation to an appropriate targeting agent. For example, in some embodiments, the targeting agent is a moiety that has affinity for an inflammatory factor (e.g., a cytokine or a cytokine receptor moiety (e.g., TNF-a receptor)). In some embodiments, the targeting agent is a sugar, peptide, antibody or antibody fragment, hormone, hormone receptor, or the like.

In some embodiments of the present invention, the targeting agent includes but is not limited to an antibody, receptor ligand, hormone, vitamin, and antigen; however, the present invention is not limited by the nature of the targeting agent. In some embodiments, the antibody is specific for a disease-specific antigen. In some embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In some embodiments, the receptor ligand is folic acid.

Antibodies can be generated to allow for the targeting of antigens or immunogens (e.g., tumor, tissue or pathogen specific antigens) on various biological targets (e.g., pathogens, tumor cells, normal tissue). Such antibodies include but are not limited to polyclonal, monoclonal, chimeric, single chain, Fab fragments, and an Fab expression library.

In some embodiments, the targeting agent is an antibody. In some embodiments, the antibodies recognize, for example, tumor-specific epitopes (e.g., TAG-72 (See, e.g., Kjeldsen et al., Cancer Res. 48:2214-2220 (1988); U.S. Pat. Nos. 5,892,020; 5,892,019; and 5,512,443; each herein incorporated by reference in their entireties); human carcinoma antigen (See, e.g., U.S. Pat. Nos. 5,693,763; 5,545,530; and 5,808,005; each herein incorporated by reference in their entireties); TP1 and TP3 antigens from osteocarcinoma cells (See, e.g., U.S. Pat. No. 5,855,866; herein incorporated by reference in its entirety); Thomsen-Friedenreich (TF) antigen from adenocarcinoma cells (See, e.g., U.S. Pat. No. 5,110,911; herein incorporated by reference in its entirety); “KC-4 antigen” from human prostrate adenocarcinoma (See, e.g., U.S. Pat. Nos. 4,708,930 and 4,743,543; each herein incorporated by reference in their entireties); a human colorectal cancer antigen (See, e.g., U.S. Pat. No. 4,921,789; herein incorporated by reference in its entirety); CA125 antigen from cystadenocarcinoma (See, e.g., U.S. Pat. No. 4,921,790; herein incorporated by reference in its entirety); DF3 antigen from human breast carcinoma (See, e.g., U.S. Pat. Nos. 4,963,484 and 5,053,489; each herein incorporated by reference in their entireties); a human breast tumor antigen (See, e.g., U.S. Pat. No. 4,939,240: herein incorporated by reference in its entirety); p97 antigen of human melanoma (See, e.g., U.S. Pat. No. 4,918,164: herein incorporated by reference in its entirety); carcinoma or orosomucoid-related antigen (CORA)(See, e.g., U.S. Pat. No. 4,914,021; herein incorporated by reference in its entirety); a human pulmonary carcinoma antigen that reacts with human squamous cell lung carcinoma but not with human small cell lung carcinoma (See, e.g., U.S. Pat. No. 4,892,935; herein incorporated by reference in its entirety); T and Tn haptens in glycoproteins of human breast carcinoma (See, e.g., Springer et al., Carbohydr. Res. 178:271-292 (1988); herein incorporated by reference in its entirety), MSA breast carcinoma glycoprotein termed (See, e.g., Tjandra et al., Br. J. Surg. 75:811-817 (1988); herein incorporated by reference in its entirety); MFGM breast carcinoma antigen (See, e.g., Ishida et al., Tumor Biol. 10:12-22 (1989); herein incorporated by reference in its entirety); DU-PAN-2 pancreatic carcinoma antigen (See, e.g., Lan et al., Cancer Res. 45:305-310 (1985); herein incorporated by reference in its entirety); CAl25 ovarian carcinoma antigen (See, e.g., Hanisch et al., Carbohydr. Res. 178:29-47 (1988); herein incorporated by reference in its entirety); YH206 lung carcinoma antigen (See, e.g., Hinoda et al., (1988) Cancer J. 42:653-658 (1988); herein incorporated by reference in its entirety).

In some embodiments, the targeting agents target the central nervous system (CNS). In some embodiments, where the targeting agent is specific for the CNS, the targeting agent is transferrin (see, e.g., Daniels, T. R., et al., Clinical Immunology, 2006. 121(2): p. 159-176; Daniels, T. R., et al., Clinical Immunology, 2006. 121(2): p. 144-158; each herein incorporated by reference in their entireties). Transferrin has been utilized as a targeting vector to transport, for example, drugs, liposomes and proteins across the blood-brain barrier (BBB) by receptor mediated transcytosis (see, e.g., Smith, M. W. and M. Gumbleton, Journal of Drug Targeting, 2006. 14(4): p. 191-214; herein incorporated by reference in its entirety). In some embodiments, the targeting agents target neurons within the central nervous system (CNS). In some embodiments, where the targeting agent is specific for neurons within the CNS, the targeting agent is a synthetic tetanus toxin fragment (e.g., a 12 amino acid peptide (Tet 1) (HLNILSTLWKYR)) (see, e.g., Liu, J. K., et al., Neurobiology of Disease, 2005. 19(3): p. 407-418; herein incorporated by reference in its entirety).

In some embodiments, the dendrimer (e.g., a glycidated dendrimer) is conjugated (e.g., directly or indirectly) (e.g., through ester linkages in a “one pot” reaction) to an imaging agent. A multiplicity of imaging agents find use in the present invention. In some embodiments, a conjugated dendrimer of the present invention (e.g., a dendrimer conjugated with one or more functional groups through, for example, initial glycidation of the dendrimer followed by simultaneous conjugation of one or more functional groups (e.g., through ester linkages in a “one pot” reaction)) comprises at least one imaging agent that can be readily imaged. The present invention is not limited by the nature of the imaging component used. In some embodiments of the present invention, imaging modules comprise surface modifications of quantum dots (See e.g., Chan and Nie, Science 281:2016 (1998)) such as zinc sulfide-capped cadmium selenide coupled to biomolecules (Sooklal, Adv. Mater., 10:1083 (1998)).

In some embodiments, once a component(s) of a targeted dendrimer (e.g., a conjugated dendrimer of the present invention (e.g., conjugated with at least a targeting agent through, for example, initial glycidation of the dendrimer followed by simultaneous conjugation of one or more functional groups (e.g., through ester linkages in a “one pot” reaction)) has attached to (or been internalized into) a target cell (e.g., tumor cell and or inflammatory cell), one or more modules serves to image its location. In some embodiments, chelated paramagnetic ions, such as Gd(III)-diethylenetriaminepentaacetic acid (Gd(III)-DTPA), are conjugated to a dendrimer. Other paramagnetic ions that may be useful in this context include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof.

Dendrimeric gadolinium contrast agents have even been used to differentiate between benign and malignant breast tumors using dynamic MRI, based on how the vasculature for the latter type of tumor images more densely (Adam et al., Ivest. Rad. 31:26 (1996)). Thus, MRI provides a particularly useful imaging system of the present invention.

Conjugated dendrimers of the present invention (e.g., dendrimers conjugated with one or more functional groups through, for example, initial glycidation of the dendrimer followed by simultaneous conjugation of one or more functional groups (e.g., through ester linkages in a “one pot” reaction)) allow functional microscopic imaging of tumors and provide improved methods for imaging. The methods find use in vivo, in vitro, and ex vivo. For example, in one embodiment, dendrimer functional groups are designed to emit light or other detectable signals upon exposure to light. Although the labeled functional groups may be physically smaller than the optical resolution limit of the microscopy technique, they become self-luminous objects when excited and are readily observable and measurable using optical techniques. In some embodiments of the present invention, sensing fluorescent biosensors in a microscope involves the use of tunable excitation and emission filters and multiwavelength sources (See, e.g., Farkas et al., SPEI 2678:200 (1997); herein incorporated by reference in its entirety). In embodiments where the imaging agents are present in deeper tissue, longer wavelengths in the Near-infrared (NMR) are used (See e.g., Lester et al., Cell Mol. Biol. 44:29 (1998); herein incorporated by reference in its entirety). Biosensors that find use with the present invention include, but are not limited to, fluorescent dyes and molecular beacons.

In some embodiments of the present invention, in vivo imaging is accomplished using functional imaging techniques. Functional imaging is a complementary and potentially more powerful techniques as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce. When excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments, biosensor-comprising pro-drug complexes are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic biosensors for pH, oxygen concentration, Ca²+ concentration, and other physiologically relevant analytes.

In some embodiments, the present invention provides dendrimers (e.g., dendrimers conjugated with one or more functional groups through, for example, initial glycidation of the dendrimer followed by simultaneous conjugation of one or more functional groups (e.g., through ester linkages in a “one pot” reaction)) having a biological monitoring component. The biological monitoring or sensing component of a dendrimer is one that can monitor the particular response in a target cell (e.g., tumor cell) induced by an agent (e.g., a therapeutic agent provided by a conjugated dendrimer). While the present invention is not limited to any particular monitoring system, the invention is illustrated by methods and compositions for monitoring cancer treatments. In preferred embodiments of the present invention, the agent induces apoptosis in cells and monitoring involves the detection of apoptosis. In some embodiments, the monitoring component is an agent that fluoresces at a particular wavelength when apoptosis occurs. For example, in a preferred embodiment, caspase activity activates green fluorescence in the monitoring component. Apoptotic cancer cells, which have turned red as a result of being targeted by a particular signature with a red label, turn orange while residual cancer cells remain red. Normal cells induced to undergo apoptosis (e.g., through collateral damage), if present, will fluoresce green.

In these embodiments, fluorescent groups such as fluorescein are employed in the imaging agent. Fluorescein is easily attached to the dendrimer surface via the isothiocyanate derivatives, available from MOLECULAR PROBES, Inc. This allows the conjugated dendrimer (e.g., a dendrimer conjugated with one or more functional groups through, for example, initial glycidation of the dendrimer followed by simultaneous conjugation of one or more functional groups (e.g., through ester linkages in a “one pot” reaction)) to be imaged with the cells via confocal microscopy. Sensing of the effectiveness of the conjugated dendrimer or components thereof is preferably achieved by using fluorogenic peptide enzyme substrates. For example, apoptosis caused by the therapeutic agent results in the production of the peptidase caspase-1 (ICE). CALBIOCHEM sells a number of peptide substrates for this enzyme that release a fluorescent moiety. A particularly useful peptide for use in the present invention is: MCA-Tyr-Glu-Val-Asp-Gly-Trp-Lys-(DNP)-NH₂ (SEQ ID NO: 1) where MCA is the (7-methoxycoumarin-4-yl)acetyl and DNP is the 2,4-dinitrophenyl group (See, e.g., Talanian et al., J. Biol. Chem., 272: 9677 (1997); herein incorporated by reference in its entirety). In this peptide, the MCA group has greatly attenuated fluorescence, due to fluorogenic resonance energy transfer (FRET) to the DNP group. When the enzyme cleaves the peptide between the aspartic acid and glycine residues, the MCA and DNP are separated, and the MCA group strongly fluoresces green (excitation maximum at 325 nm and emission maximum at 392 nm). In some embodiments, the lysine end of the peptide is linked to pro-drug complex, so that the MCA group is released into the cytosol when it is cleaved. The lysine end of the peptide is a useful synthetic handle for conjugation because, for example, it can react with the activated ester group of a bifunctional linker such as Mal-PEG-OSu. Thus the appearance of green fluorescence in the target cells produced using these methods provides a clear indication that apoptosis has begun (if the cell already has a red color from the presence of aggregated quantum dots, the cell turns orange from the combined colors).

Additional fluorescent dyes that find use with the present invention include, but are not limited to, acridine orange, reported as sensitive to DNA changes in apoptotic cells (see, e.g., Abrams et al., Development 117:29 (1993); herein incorporated by reference in its entirety) and cis-parinaric acid, sensitive to the lipid peroxidation that accompanies apoptosis (see, e.g., Hockenbery et al., Cell 75:241 (1993); herein incorporated by reference in its entirety). It should be noted that the peptide and the fluorescent dyes are merely exemplary. It is contemplated that any peptide that effectively acts as a substrate for a caspase produced as a result of apoptosis finds use with the present invention.

In some embodiments, conjugation between a dendrimer (e.g., terminal arm of a dendrimer) and a functional group or between functional groups is accomplished through use of a 1,3-dipolar cycloaddition reaction (“click chemistry”). ‘Click chemistry’ involves, for example, the coupling of two different moieties (e.g., a therapeutic agent and a functional group) (e.g., a first functional group and a second functional group) via a 1,3-dipolar cycloaddition reaction between an alkyne moiety (or equivalent thereof) on the surface of the first moeity and an azide moiety (e.g., present on a triazine composition of the present invention) (or equivalent thereof) (or any active end group such as, for example, a primary amine end group, a hydroxyl end group, a carboxylic acid end group, a thiol end group, etc.) on the second moiety. ‘Click’ chemistry is an attractive coupling method because, for example, it can be performed with a wide variety of solvent conditions including aqueous environments. For example, the stable triazole ring that results from coupling the alkyne with the azide is frequently achieved at quantitative yields and is considered to be biologically inert (see, e.g., Rostovtsev, V. V.; et al., Angewandte Chemie-International Edition 2002, 41, (14), 2596; Wu, P.; et al., Angewandte Chemie-International Edition 2004, 43, (30), 3928-3932; each herein incorporated by reference in their entireties).

The present invention is not limited by the type of therapeutic agent delivered via conjugated dendrimers (e.g., dendrimers conjugated with one or more functional groups (e.g., one or more therapeutic agents) through, for example, initial glycidation of the dendrimer followed by simultaneous conjugation of one or more functional groups (e.g., through ester linkages in a “one pot” reaction)) of the present invention. For example, a therapeutic agent may be any agent selected from the group comprising, but not limited to, autoimmune disorder agent and/or an inflammatory disorder agent. Additional examples of therapeutic agents include, but are not limited to, a pain relief agent, a pain relief agent antagonist, a chemotherapeutic agent, an anti-oncogenic agent, an anti-angiogenic agent, a tumor suppressor agent, an anti-microbial agent, or an expression construct comprising a nucleic acid encoding a therapeutic protein.

It is contemplated that components of conjugated dendrimers of the present invention (e.g., dendrimers conjugated with one or more functional groups through, for example, initial glycidation of the dendrimer followed by simultaneous conjugation of one or more functional groups (e.g., through ester linkages in a “one pot” reaction)) provide therapeutic benefits to patients suffering from medical conditions and/or diseases (e.g., cancer, inflammatory disease, chronic pain, autoimmune disease, etc.).

Indeed, in some embodiments of the present invention, methods and compositions are provided for the treatment of inflammatory diseases (e.g., dendrimers conjugated with therapeutic agents configured for treating inflammatory diseases). Inflammatory diseases include but are not limited to arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate dihydrate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, and yersinial arthritis.

In some embodiments, the conjugated dendrimers of the present invention (e.g., dendrimers conjugated with one or more functional groups through, for example, initial glycidation of the dendrimer followed by simultaneous conjugation of one or more functional groups (e.g., through ester linkages in a “one pot” reaction)) configured for treating autoimmune disorders and/or inflammatory disorders (e.g., rheumatoid arthritis) are co-administered to a subject (e.g., a human suffering from an autoimmune disorder and/or an inflammatory disorder) a therapeutic agent configured for treating autoimmune disorders and/or inflammatory disorders (e.g., rheumatoid arthritis). Examples of such agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone).

In some embodiments, the medical condition and/or disease is pain (e.g., chronic pain, mild pain, recurring pain, severe pain, etc.). In some embodiments, the conjugated dendrimers of the present invention (e.g., dendrimers conjugated with one or more functional groups through, for example, initial glycidation of the dendrimer followed by simultaneous conjugation of one or more functional groups (e.g., through ester linkages in a “one pot” reaction)) are configured to deliver pain relief agents to a subject. In some embodiments, the dendrimer conjugates are configured to deliver pain relief agents and pain relief agent antagonists to counter the side effects of pain relief agents. The dendrimer conjugates are not limited to treating a particular type of pain and/or pain resulting from a disease. Examples include, but are not limited to, pain resulting from trauma (e.g., trauma experienced on a battlefield, trauma experienced in an accident (e.g., car accident)). In some embodiments, the dendrimer conjugates of the present invention are configured such that they are readily cleared from the subject (e.g., so that there is little to no detectable toxicity at efficacious doses).

In some embodiments, the disease is cancer. The present invention is not limited by the type of cancer treated using the compositions and methods of the present invention. Indeed, a variety of cancer can be treated including, but not limited to, prostate cancer, colon cancer, breast cancer, lung cancer and epithelial cancer. Similarly, the present invention is not limited by the type of inflammatory disease and/or chronic pain treated using the compositions of the present invention. Indeed, a variety of diseases can be treated including, but not limited to, arthritis (e.g., osteoarthritis, rheumatoid arthritis, etc.), inflammatory bowel disease (e.g., colitis, Crohn's disease, etc.), autoimmune disease (e.g., lupus erythematosus, multiple sclerosis, etc.), inflammatory pelvic disease, etc.

In some embodiments, the disease is a neoplastic disease, selected from, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma. In some embodiments, the disease is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome. In some embodiments, the disease is a viral disease selected from the group consisting of, but not limited to, viral disease caused by hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

The present invention also includes methods involving co-administration of the conjugated dendrimers of the present invention (e.g., dendrimers conjugated with one or more functional groups through, for example, initial glycidation of the dendrimer followed by simultaneous conjugation of one or more functional groups (e.g., through ester linkages in a “one pot” reaction)) with one or more additional active agents. Indeed, it is a further aspect of this invention to provide methods for enhancing prior art therapies and/or pharmaceutical compositions by co-administering conjugated dendrimers of this invention. In co-administration procedures, the agents may be administered concurrently or sequentially. In some embodiments, the conjugated dendrimers described herein are administered prior to the other active agent(s). The agent or agents to be co-administered depends on the type of condition being treated. For example, when the condition being treated is arthritis, the additional agent can be an agent effective in treating arthritis (e.g., TNF-a inhibitors such as anti-TNF α monoclonal antibodies (such as REMICADE®, CDP-870 and HUMIRA™ (adalimumab) and TNF receptor-immunoglobulin fusion molecules (such as ENBREL®) (entanercept), IL-1 inhibitors, receptor antagonists or soluble IL-1Rα (e.g. KINERETT™ or ICE inhibitors), nonsteroidal anti-inflammatory agents (NSAIDS), piroxicam, diclofenac, naproxen, flurbiprofen, fenoprofen, ketoprofen ibuprofen, fenamates, mefenamic acid, indomethacin, sulindac, apazone, pyrazolones, phenylbutazone, aspirin, COX-2 inhibitors (such as CELEBREX® (celecoxib), VIOXX® (rofecoxib), BEXTRA® (valdecoxib) and etoricoxib, (preferably MMP-13 selective inhibitors), NEUROTIN®, pregabalin, sulfasalazine, low dose methotrexate, leflunomide, hydroxychloroquine, d-penicillamine, auranofin or parenteral or oral gold). The additional agents to be co-administered can be any of the well-known agents in the art, including, but not limited to, those that are currently in clinical use. The determination of appropriate type and dosage of radiation treatment is also within the skill in the art or can be determined with relative ease.

In some embodiments, the composition is co-administered with an anti-cancer agent (e.g., Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-zitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; CI-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda). Other anti-cancer agents include, but are not limited to, Antiproliferative agents (e.g., Piritrexim Isothionate), Antiprostatic hypertrophy agent (e.g., Sitogluside), Benign prostatic hyperplasia therapy agents (e.g., Tamsulosin Hydrochloride), Prostate growth inhibitor agents (e.g., Pentomone), and Radioactive agents: Fibrinogen I 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; and Triolein I 131).

Additional anti-cancer agents include, but are not limited to anti-cancer Supplementary Potentiating Agents: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. Still other anticancer agents include, but are not limited to, annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-Cl-2′ deoxyadenosine; Fludarabine-PO₄; mitoxantrone; mitozolomide; Pentostatin; and Tomudex. One particularly preferred class of anticancer agents is taxanes (e.g., paclitaxel and docetaxel). Another important category of anticancer agent is annonaceous acetogenin.

In some embodiments, the composition is co-administered with a pain relief agent. In some embodiments, the pain relief agents include, but are not limited to, analgesic drugs, anxiolytic drugs, anesthetic drugs, antipsychotic drugs, hypnotic drugs, sedative drugs, and muscle relaxant drugs.

In some embodiments, the analgesic drugs include, but are not limited to, non-steroidal anti-inflammatory drugs, COX-2 inhibitors, and opiates. In some embodiments, the non-steroidal anti-inflammatory drugs are selected from the group consisting of Acetylsalicylic acid (Aspirin), Amoxiprin, Benorylate/Benorilate, Choline magnesium salicylate, Diflunisal, Ethenzamide, Faislamine, Methyl salicylate, Magnesium salicylate, Salicyl salicylate, Salicylamide, arylalkanoic acids, Diclofenac, Aceclofenac, Acemethacin, Alclofenac, Bromfenac, Etodolac, Indometacin, Nabumetone, Oxametacin, Proglumetacin, Sulindac, Tolmetin, 2-arylpropionic acids, Ibuprofen, Alminoprofen, Benoxaprofen, Carprofen, Dexibuprofen, Dexketoprofen, Fenbufen, Fenoprofen, Flunoxaprofen, Flurbiprofen, Ibuproxam, Indoprofen, Ketoprofen, Ketorolac, Loxoprofen, Naproxen, Oxaprozin, Pirprofen, Suprofen, Tiaprofenic acid), N-arylanthranilic acids, Mefenamic acid, Flufenamic acid, Meclofenamic acid, Tolfenamic acid, pyrazolidine derivatives, Phenylbutazone, Ampyrone, Azapropazone, Clofezone, Kebuzone, Metamizole, Mofebutazone, Oxyphenbutazone, Phenazone, Sulfinpyrazone, oxicams, Piroxicam, Droxicam, Lornoxicam, Meloxicam, Tenoxicam, sulphonanilides, nimesulide, licofelone, and omega-3 fatty acids. In some embodiments, the COX-2 inhibitors are selected from the group consisting of Celecoxib, Etoricoxib, Lumiracoxib, Parecoxib, Rofecoxib, and Valdecoxib. In some embodiments, the opiate drugs are selected from the group consisting of natural opiates, alkaloids, morphine, codeine, thebaine, semi-synthetic opiates, hydromorphone, hydrocodone, oxycodone, oxymorphone, desomorphine, diacetylmorphine (Heroin), nicomorphine, dipropanoylmorphine, diamorphine, benzylmorphine, Buprenorphine, Nalbuphine, Pentazocine, meperidine, diamorphine, ethylmorphine, fully synthetic opioids, fentanyl, pethidine, Oxycodone, Oxymorphone, methadone, tramadol, Butorphanol, Levorphanol, propoxyphene, endogenous opioid peptides, endorphins, enkephalins, dynorphins, and endomorphins.

In some embodiments, the anxiolytic drugs include, but are not limited to, benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze, Triazolam, serotonin 1A agonists, Buspirone (BuSpar), barbituates, amobarbital (Amytal), pentobarbital (Nembutal), secobarbital (Seconal), Phenobarbital, Methohexital, Thiopental, Methylphenobarbital, Metharbital, Barbexaclone), hydroxyzine, cannabidiol, valerian, kava (Kava Kava), chamomile, Kratom, Blue Lotus extracts, Sceletium tortuosum (kanna) and bacopa monniera.

In some embodiments, the anesthetic drugs include, but are not limited to, local anesthetics, procaine, amethocaine, cocaine, lidocaine, prilocalne, bupivacaine, levobupivacaine, ropivacaine, dibucaine, inhaled anesthetics, Desflurane, Enflurane, Halothane, Isoflurane, Nitrous oxide, Sevoflurane, Xenon, intravenous anesthetics, Barbiturates, amobarbital (Amytal), pentobarbital (Nembutal), secobarbital (Seconal), Phenobarbital, Methohexital, Thiopental, Methylphenobarbital, Metharbital, Barbexaclone)), Benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, Etomidate, Ketamine, and Propofol.

In some embodiments, the antipsychotic drugs include, but are not limited to, butyrophenones, haloperidol, phenothiazines, Chlorpromazine (Thorazine), Fluphenazine (Prolixin), Perphenazine (Trilafon), Prochlorperazine (Compazine), Thioridazine (Mellaril), Trifluoperazine (Stelazine), Mesoridazine, Promazine, Triflupromazine (Vesprin), Levomepromazine (Nozinan), Promethazine (Phenergan)), thioxanthenes, Chlorprothixene, Flupenthixol (Depixol and Fluanxol), Thiothixene (Navane), Zuclopenthixol (Clopixol & Acuphase)), clozapine, olanzapine, Risperidone (Risperdal), Quetiapine (Seroquel), Ziprasidone (Geodon), Amisulpride (Solian), Paliperidone (Invega), dopamine, bifeprunox, norclozapine (ACP-104), Aripiprazole (Abilify), Tetrabenazine, and Cannabidiol.

In some embodiments, the hypnotic drugs include, but are not limited to, Barbiturates, Opioids, benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, nonbenzodiazepines, Zolpidem, Zaleplon, Zopiclone, Eszopiclone, antihistamines, Diphenhydramine, Doxylamine, Hydroxyzine, Promethazine, gamma-hydroxybutyric acid (Xyrem), Glutethimide, Chloral hydrate, Ethchlorvynol, Levomepromazine, Chlormethiazole, Melatonin, and Alcohol.

In some embodiments, the sedative drugs include, but are not limited to, barbituates, amobarbital (Amytal), pentobarbital (Nembutal), secobarbital (Seconal), Phenobarbital, Methohexital, Thiopental, Methylphenobarbital, Metharbital, Barbexaclone), benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, herbal sedatives, ashwagandha, catnip, kava (Piper methysticum), mandrake, marijuana, valerian, solvent sedatives, chloral hydrate (Noctec), diethyl ether (Ether), ethyl alcohol (alcoholic beverage), methyl trichloride (Chloroform), nonbenzodiazepine sedatives, eszopiclone (Lunesta), zaleplon (Sonata), zolpidem (Ambien), zopiclone (Imovane, Zimovane)), clomethiazole (clomethiazole), gamma-hydroxybutyrate (GHB), Thalidomide, ethchlorvynol (Placidyl), glutethimide (Doriden), ketamine (Ketalar, Ketaset), methaqualone (Sopor, Quaalude), methyprylon (Noludar), and ramelteon (Rozerem).

In some embodiments, the muscle relaxant drugs include, but are not limited to, depolarizing muscle relaxants, Succinylcholine, short acting non-depolarizing muscle relaxants, Mivacurium, Rapacuronium, intermediate acting non-depolarizing muscle relaxants, Atracurium, Cisatracurium, Rocuronium, Vecuronium, long acting non-depolarizing muscle relaxants, Alcuronium, Doxacurium, Gallamine, Metocurine, Pancuronium, Pipecuronium, and d-Tubocurarine.

In some embodiments, the composition is co-administered with a pain relief agent antagonist. In some embodiments, the pain relief agent antagonists include drugs that counter the effect of a pain relief agent (e.g., an anesthetic antagonist, an analgesic antagonist, a mood stabilizer antagonist, a psycholeptic drug antagonist, a psychoanaleptic drug antagonist, a sedative drug antagonist, a muscle relaxant drug antagonist, and a hypnotic drug antagonist). In some embodiments, pain relief agent antagonists include, but are not limited to, a respiratory stimulant, Doxapram, BIMU-8, CX-546, an opiod receptor antagonist, Naloxone, naltrexone, nalorphine, levallorphan, cyprodime, naltrindole, norbinaltorphimine, buprenorphine, a benzodiazepine antagonist, flumazenil, a non-depolarizing muscle relaxant antagonist, and neostigmine.

Where clinical applications are contemplated, in some embodiments of the present invention, the conjugated dendrimers (e.g., dendrimers conjugated with one or more functional groups through, for example, initial glycidation of the dendrimer followed by simultaneous conjugation of one or more functional groups (e.g., through ester linkages in a “one pot” reaction)) are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight dendrimer formulation may be administered using one or more of the routes described herein.

In preferred embodiments, the conjugated dendrimers of the present invention (e.g., dendrimers conjugated with one or more functional groups through, for example, initial glycidation of the dendrimer followed by simultaneous conjugation of one or more functional groups (e.g., through ester linkages in a “one pot” reaction)) are used in conjunction with appropriate salts and buffers to render delivery of the compositions in a stable manner to allow for uptake by target cells. Buffers also are employed when the conjugated dendrimers are introduced into a patient. Aqueous compositions comprise an effective amount of the conjugated dendrimers to cells dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with vectors, cells, or tissues, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The conjugated dendrimers (e.g., dendrimers conjugated with one or more functional groups through, for example, initial glycidation of the dendrimer followed by simultaneous conjugation of one or more functional groups (e.g., through ester linkages in a “one pot” reaction)) may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

In some embodiments, a therapeutic agent is released from conjugated dendrimers (e.g., dendrimers conjugated with one or more functional groups through, for example, initial glycidation of the dendrimer followed by simultaneous conjugation of one or more functional groups (e.g., through ester linkages in a “one pot” reaction)) within a target cell (e.g., within an endosome). This type of intracellular release (e.g., endosomal disruption of a linker-therapeutic conjugate) is contemplated to provide additional specificity for the compositions and methods of the present invention. The present invention provides dendrimers with multiple (e.g., 100-150) reactive sites for the conjugation of linkers and/or functional groups comprising, but not limited to, therapeutic agents, targeting agents, imaging agents and biological monitoring agents.

The compositions and methods of the present invention are contemplated to be equally effective whether or not the conjugated dendrimers (e.g., dendrimers conjugated with one or more functional groups through, for example, initial glycidation of the dendrimer followed by simultaneous conjugation of one or more functional groups (e.g., through ester linkages in a “one pot” reaction)) comprise a fluorescein (e.g. FITC) imaging agent. Thus, each functional group present in a dendrimer composition is able to work independently of the other functional groups. Thus, the present invention provides conjugated dendrimers that can comprise multiple combinations of targeting, therapeutic, imaging, and biological monitoring functional groups.

The present invention also provides a very effective and specific method of delivering molecules (e.g., therapeutic and imaging functional groups) to the interior of target cells (e.g., cancer cells). Thus, in some embodiments, the present invention provides methods of therapy that comprise or require delivery of molecules into a cell in order to function (e.g., delivery of genetic material such as siRNAs).

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the conjugated dendrimers in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, conjugated dendrimers (e.g., dendrimers conjugated with one or more functional groups through, for example, initial glycidation of the dendrimer followed by simultaneous conjugation of one or more functional groups (e.g., through ester linkages in a “one pot” reaction)) are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The conjugated dendrimers also may be formulated as inhalants for the treatment of lung cancer and such like.

The conjugated dendrimers (e.g., dendrimers conjugated with one or more functional groups through, for example, initial glycidation of the dendrimer followed by simultaneous conjugation of one or more functional groups (e.g., through ester linkages in a “one pot” reaction)) may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC) size exclusion chromatography (SEC) (equipped with multi-angle laser light scattering, dual UV and refractive index detectors), capillary electrophoresis and get electrophoresis. These analytical methods assure the uniformity of the dendrimer population and are important in the quality control of dendrimer production for eventual use in in vivo applications. Most importantly, extensive work has been performed with dendrimers showing no evidence of toxicity when administered intravenously (Roberts et al., J. Biomed. Mater. Res., 30:53 (1996) and Boume et al., J. Magnetic Resonance Imaging, 6:305 (1996)).

In some embodiments, the present invention also provides kits comprising one or more of the reagents and tools necessary to generate a dendrimer comprising one or more ligands (e.g., functional groups) wherein each ligand is attached to the dendrimer by an ester bond. Examples of such reagents include, but are not limited to, dendrimers (e.g., a polyamideamine (PAMAM) dendrimer, a polypropylamine (POPAM) dendrimer, and a PAMAM-POPAM dendrimer. In some embodiments, the dendrimer is a Baker-Huang PAMAM dendrimer) not having undergone glycidation, one or more ligands (e.g., therapeutic agents, targeting agents, trigger agents, and imaging agents), and any reagents necessary for conjugation of such ligands with such dendrimers. In some embodiments, the kit comprises a vessel designed to accommodate the one-pot dendrimer synthesis methods of the present invention (e.g., methods for synthesizing multifunctional dendrimers (e.g., dendrimers conjugated with one or more functional groups) through, for example, initial glycidation of a dendrimer followed by simultaneous conjugation of one or more functional groups (e.g., through ester linkages in a “one pot” reaction)).

### EXPERIMENTAL

The following examples are provided in order to demonstrate and further illustrate certain preferred embodiments and aspects of the present invention and are not to be construed as limiting the scope thereof.

### Example 1

Previous experiments involving dendrimer related technologies are located in U.S. Pat. Nos. 6,471,968, 7,078,461, and U.S. patent application Ser. Nos. 09/940,243, 10/431,682, 11,503,742, 11,661,465, 11/523,509, 12/403,179, 12/106,876, 11/827,637, U.S. Provisional Patent Application Ser. Nos. 61/140,840, 61/091,608, 61/097,780, 61/101,461, 61/251,244; each herein incorporated by reference in their entireties.

### Example 2

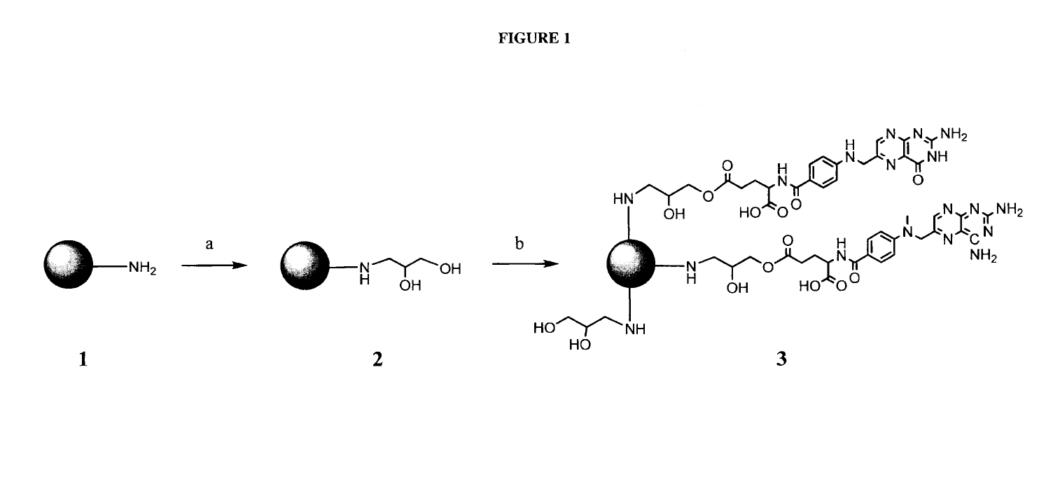

A targeted nanodendrimeric anticancer prodrug conjugate of MTX, FA with PAMAM dendrimer was prepared according to the synthetic scheme outlined in FIG. 1 (Zhang et al. (2010) Bioconjugate Chem. 21:489-495; herein incorporated by reference in its entirety). Beginning with dendrimer 1, the amino group of 1 attached the three-member ring of glycidol, ethylene oxide group, in methanol at room temperature under nitrogen overnight to form hydroxyl-terminated dendrimer 2. The free amino groups on the surface of dendrimer 1 were fully capped by 2,3-dihydroxylpropyl groups. 2 was purified either by dialysis with cellulose dialysis membrane against water, or buffer and then water, or isotonic saline solution and then water. In some trials, the purification was performed also by precipitation process in organic solvents such as diethyl ether, hexane, cyclohexane, ethyl acetate, acetone, chloroform, dichloromethane, tetrahydrofuran, or any combination solution of aforementioned solvents, or any combination solution of aforementioned solvents and more polar solvents such as dioxane, ethanol, methanol, N,N-dimethylformamide, dimethylsulfoxide, and N,N-dimethylacetamide. The MTX and FA were attached to the hydroxyl groups of hydroxyl-terminated dendrimer 2 through ester bonds with coupling agents such as 2-chloro-1-methylpyridinium iodide and 4-(dimethylamino)pyridine, or dicyclohexylcarbodiimide and 4-(dimethylamino)-pyridine or diethyl azodicarboxylate and triphenylphosphine, or any other coupling agents for ester bond formation. Second step is a one-pot reaction. The reaction was conducted in N,N-dimethylformamide, or dimethylsulfoxide, or N,N-dimethylacetamide or any combination of the solvents under nitrogen at room temperature for about 24 hours. The final product, conjugate 3, was purified by dialysis with cellulose dialysis membrane against buffer and then water, or saline and then water or water only. The hydroxyl-terminated dendrimer 2 and conjugate 3 were characterized by ¹H NMR, MALDI-TOF, GPC, and HPLC.

### Example 3

### Synthesis of Hydroxyl-Terminated G5 PAMAM Dendrimer (Method 1)

G5 PAMAM dendrimer (200 mg) was dissolved in 10 mL of methanol in a 25 mL flask. To the solution was added glycidol (127 μL). The mixture was stirred at room temperature under nitrogen over night. The reaction mixture was added to diethyl ether (50 mL) with stirring for 30 minutes. The mixture was centrifuged and supernatant was removed. The product was then suspended in diethyl ether (50 mL) with stirring for 30 minutes. The mixture was centrifuged and supernatant was removed. The final product was dried by vacuum at room temperature for 72 hours to yield 266 mg of hydroxyl-terminated G5 PAMAM dendrimer.

### Example 4

### Synthesis of Hydroxyl-Terminated G5 PAMAM Dendrimer (Method 2)

G5 PAMAM dendrimer (500 mg) was dissolved in 20 mL of methanol in a 50 mL flask. To the solution was added glycidol (442 mg, 396 μL). The mixture was stirred at room temperature under nitrogen over night. The methanol was removed by a rotavapor at room temperature. The mixture was dialyzed against water (4 times 4 L) with cellulose dialysis membrane (MWCO=1,000) for 48 hours, and then dried by lyophilization (3 days) to yield 677 mg of hydroxyl-terminated G5 PAMAM dendrimer.

### Example 5

### Synthesis of Conjugate of G5 PAMAM Dendrimer, Folic Acid, and Methotrexate

### Method 1

A solution of hydroxyl-terminated G5 PAMAM dendrimer (200 mg), MTX (22.72 mg, 0.05 mmol), and FA (15.44 mg, 0.035 mmol) in dimethyl sulfoxide (10 mL) was stirred at room temperature under nitrogen. To the solution was added 4-(dimethylamino)-pyridine (24.44 mg, 0.2 mmol) and 2-chloro-1-methylpyridine iodide (25.59 mg, 0.1 mmol). The mixture was stirred at room temperature under nitrogen overnight, and then added into diethyl ether (60 mL) with stirring. The white precipitate was collected by centrifuge. The product was dissolved in water and further purified by dialysis against water (3 times 4 L) with cellulose dialysis membrane (MWCO=10,000) for 24 hours, and then dried by lyophilization (3 days) to yield 110 mg of conjugate of G5 PAMAM dendrimer, FA, and MTX.

### Example 6

### Synthesis of Conjugate of G5 PAMAM Dendrimer, Folic Acid, and Methotrexate

### Method 2

A solution of hydroxyl-terminated G5 PAMAM dendrimer (200 mg), MTX (34.08 mg, 0.075 mmol), and FA (17.66 mg, 0.04 mmol) in dimethyl sulfoxide (15 mL) was stirred at room temperature under nitrogen. To the solution was added 2-chloro-1-methylpyridine iodide (35.26 mg, 0.138 mmol) and 4-(dimethylamino)pyridine (33.72 mg, 0.276 mmol). The mixture was stirred under nitrogen at room temperature overnight and then dissolved in water (75 mL). The product was purified by dialysis against isotonic phosphate buffered saline (PBS) buffer (3 times 4 L) and then water (3 times 4 L) with cellulose dialysis membrane (MWCO=10,000) over 48 hours. The final product was dried by lyophilization (3 days) to yield 231 mg of conjugate of G5 PAMAM dendrimer, FA, and MTX.

### Example 7

### Synthesis of Conjugate of G5 Dendrimer, Folic Acid, Methotrexate, and Fluorescein

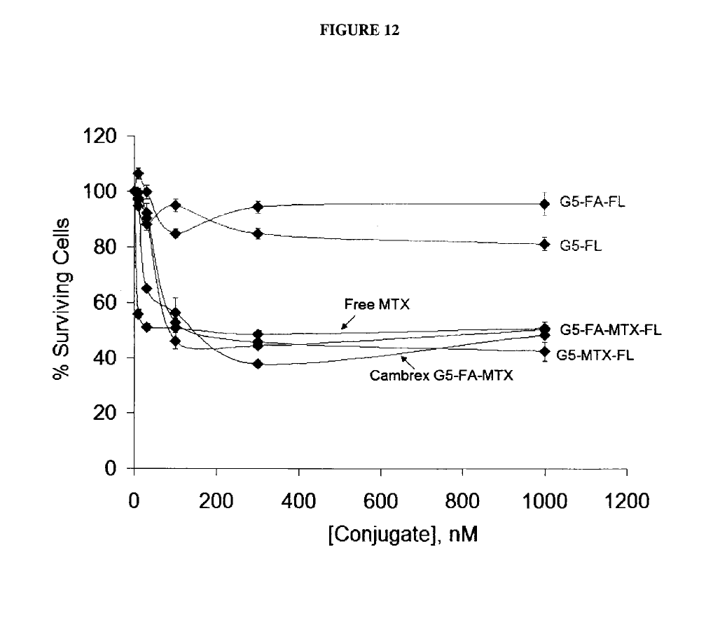

The reaction conducted is shown in FIG. 12. A solution of hydroxyl-terminated G5 dendrimer (100 mg, 0.0025 mmol), methotrexate (11.36 mg, 0.025 mmol), folic acid (5.52 mg, 0.0125 mmol), and fluorescein-5(6)-carboxamidohexanoic acid (6.80 mg, 0.0125 mmol) in dimethyl sulfoxide (10 mL) was stirred at room temperature under nitrogen. To the solution was added 2-chloro-1-methylpyridinium iodide (19.20 mg, 0.075 mmol) and 4-(dimethylamino)pyridine (18.32 mg, 0.15 mmol). The mixture was stirred overnight and then dissolved in di-water (50 mL). The product was purified by dialysis against PBS buffer (3×4 L) and then water (3 times 4 L) over 48 hours. The final product was dried by lyophilization (3 days) to yield 120 mg of the conjugate.

### Example 8

### Synthesis of Conjugate of G5 Dendrimer, Folic Acid and 7-ethyl-10-hydroxycamptothecin (SN-38)

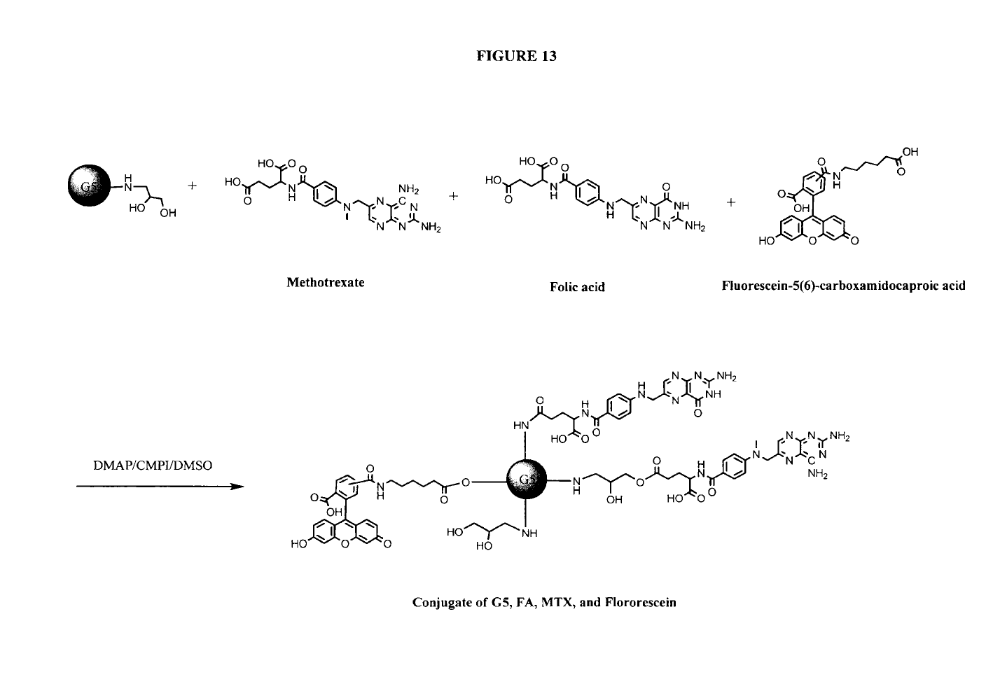

The reaction conducted is shown in FIG. 13. A solution of hydroxyl-terminated G5 dendrimer (100 mg, 0.0025 mmol), SN38-10-acetic acid (17.0 mg, 0.0375 mmol), and folic acid (8.83 mg, 0.02 mmol) in dimethyl sulfoxide (10 mL) was stirred at room temperature under nitrogen. To the solution was added 2-chloro-1-methylpyridinium iodide (17.63 mg, 0.069 mmol) and 4-(dimethylamino)pyridine (16.86 mg, 0.0.138 mmol). The mixture was stirred overnight and then dissolved in di-water (50 mL). The product was purified by dialysis against PBS buffer (3×4 L) and then water (3 times 4 L) over 48 hours. The final product was dried by lyophilization (3 days) to yield 125 mg of the conjugate.

### Example 9

The following Example describes methods of characterization and analysis of functionalized dendrimers using a “one-pot” synthesis technique as described in Examples 2-8. Dendrimer conjugates were analyzed using ¹H NMR, MALDI-TOF, HPLC, and gel permeation chromatography. Furthermore, dendrimer conjugates were functionally analyzed by determining measurements of cell binding and cytotoxicity relative to dendrimer conjugates not synthesized using “one-pot” techniques.

Materials. Amine-terminated G5-PAMAM dendrimer was purchased from Dendritech, Inc. (Midland, Mich., USA) and characterized at the Michigan Nanotechnology Institute for Medicine and Biological Science, University of Michigan. All chemicals were purchased from Sigma-Aldrich and used as received, unless otherwise specified. Water used in all experiments was purified by a Mili-Q Plus 185 water purification system (Millipore, Bedford, Mass.) with resistivity higher than 18 MΩcm. The Spectra Por dialysis membranes (MWCO 1,000 and MWCO 10,000) and phosphate buffer saline were acquired from Fisher.

Nuclear Magnetic Resonance Spectrometry. ¹H NMR spectra were recorded on a 400 MHz Varian Inova 400 nuclear magnetic spectrometer in dimethyl sulfoxide (DMSO-d₆).

Matrix-assisted Laser Desorption Ionization-Time of Flight (MALDI-TOF) Mass Spectrometry. MALDI-TOF mass spectra were recorded on a Waters TofSpec-2E spectrometer (Beverly, Mass., USA), running in linear mode with the high mass PAD detector. 2,5-dihydroxybenzoic acid (DHB) in acetonitrile/water (50:50, v/v) was used as the matrix. The instrument was calibrated with bovine serum albumin (BSA, M_{w}=66.43×10³) in sinapic acid. The data were acquired and processed by MassLynx 3.5 software.

High-Performance Liquid Chromatography (HPLC). A reverse-phase HPLC instrument consisting of a System GOLD 126 solvent module, a model 507 auto sampler equipped with a 100 μL loop and a model 166 UV detector (Beckman Coulter, Fullerton, Calif. USA) and a Phenomenex (Torrance, Calif., USA) Jupiter C5 silica-based HPLC column (250×4.6 mm, 300 Å) were used for analysis of the products. Two Phenomenex safety guards were installed upstream of the HPLC column. The mobile phase for elution of PAMAM dendrimer products was a linear gradient beginning with 100:0 water/acetonitrile (ACN) (both containing 0.14 wt % TFA) at a flow rate of 1 mL/min, reaching 20:80 (or 50:50) within 35 min. Trifluoroacetic acid (TFA) (0.14 wt % in both water and ACN) was used as a counter-ion to make the dendrimer-conjugate surface hydrophobic. All samples were dissolved in the aqueous mobile phase (water containing 0.14% TFA). The injection volume in each case was 35 μL with a sample concentration of 1 mg/mL and the detection wavelength was 210 or 280 nm. The analysis was performed using Beckman's System GOLD Nouveau software.

Gel Permeation Chromatography (GPC). GPC was used to evaluate the molecular weights and molecular weight distribution of G5-PAMAM dendrimer, dendrimer derivatives and conjugates. GPC experiments were performed on an Alliance Waters 2690 separation module (Waters Corp., Milford, Mass.) equipped with a Waters 2487 dual wavelength UV absorbance detector (Waters Corp.), a Wyatt Dawn HELEOS light scattering detector (Wyatt Technology Corp., Santa Barbara, Calif.), an Optilab rEX differential refractometer (Wyatt Technology Corp.), and TosoHaas TSK-Gel Guard PHW 06762 (75×7.5 mm, 12 μm), G 2000 PW 05761 (300×7.5 mm, 10 μm), G 3000 PW 05762 (300×7.5 mm, 10 μm), and G 4000 PW (300×7.5 mm, 17 μm) columns. Column temperature was maintained at 25±0.2° C. with a Waters temperature control module. Citric acid buffer (0.1 M) with 0.025% sodium azide in water, pH 2.74, was used as a mobile phase at a flow rate of 1 mL/min. The sample concentration was 2 mg/mL with an injection volume of 100 μL. The number average molecular weight, Mₙ, weight average molecular weight M_{w} and polydispersity index (PDI) were calculated using ASTRA 5.34.14 software (Wyatt Technology Corp.)

### Synthesis of Hydroxyl-Terminated G5 PAMAM Dendrimer (G5-Gly-OH)2

Hydroxyl-terminated G5 PAMAM dendrimer was synthesized as described in Example 4 above.

### Synthesis of Conjugate of G5 PAMAM Dendrimer, Folic Acid and Methotrexate (G5-FA-MTX)

G5-FA-MTX was synthesized as described in Example 6 above.

Measurement of cellular binding and cytotoxicity. KB cells, a sub-line of the cervical carcinoma HeLa cells (ATCC, Manassas, Va., USA), were grown as a monolayer cell culture at 37° C. and 5% CO₂ in FA-deficient RPMI 1640 medium supplemented with 10% fetal bovine serum (FBS). The 10% FBS provided an FA concentration equivalent to that present in the human serum (˜20 nM).

For assessment of the cellular binding of the synthesized conjugate, analysis was performed of the competition of the conjugate with another previously synthesized dendrimer conjugate (“G5-FI-FA-MTX”) that contained 4 molecules of the fluorescent dye fluorescein isothiocyanate (FI), 5 molecules of FA and 7 molecules of MTX (Thomas et al. (2005) J. Med. Chem. 48:3729-3735; herein incorporated by reference in its entirety). KB cells plated in 24-well plates were treated with a mixture of 100 nM of the G5-FI-FA-MTX and varying concentration of the newly synthesized G5-FA-MTX, added as mixture at the same time. The cells were incubated at 37° C. for 1 h and the FL1 fluorescence of 10,000 cells were determined by flow cytometry, as described previously (Thomas et al. (2005) J. Med. Chem. 48:3729-3735; herein incorporated by reference in its entirety).

For cytotoxicity experiments, the cells were seeded in 96-well microtiter plates (3000 cells/well) in medium containing dialyzed serum. Two days after plating, the cells were treated with the dendrimer conjugates in tissue culture medium under the conditions indicated below. A colorimetric ‘XTT’ (sodium 3-[1-(phenylaminocarbonyl)-3,4-tetrazolium]-bis(4-methoxy-6-nitro) benzene sulfonicacid hydrate) assay (Roche Molecular Biochemicals, Indianapolis, Ind.), was performed following the vendor's protocol. After incubation with the XTT labeling mixture, the microtiter plates were read on an ELISA reader (Synergy HT, BioTek) at 492 nm with the reference wavelength at 690 nm (Thomas et al. (2005) J. Med. Chem. 48:3729-3735; herein incorporated by reference in its entirety).

Results

Synthesis

G5-NH₂ 1 was used as starting material to synthesize the targeted nanodendrimeric anticancer drug G5-FA-MTX 3, FIG. 1. The average number of available primary amino groups on the surface of G5-NH₂ is 110.(23) Starting with G5-NH₂ 1, the amino groups of G5-NH₂ react with glycidol to yield G5-Gly-OH 2. In this reaction, excess glycidol was used (glycidol:NH₂=3:1) to fully cap the free amino groups on the surface of 1. While not limited to a particular mechanism, each amino group can theoretically react with two molecules of glycidol to form a tertiary amine. However, analysis indicated that the hydroxylation reaction was incomplete, which is consistent with results reported in the literature (Shi et al. (2006) Colloids and Surfaces a-Physiochemical and Engineering Aspects 272:139-150; herein incorporated by reference in its entirety. While not limited to a particular mechanism, steric hindrance prevented some of the terminal amines residing inside the dendrimer from being available for hydroxylation, resulting in an incomplete reaction (Maiti et al. (2004) Macromolecules 37:6236-6254; herein incorporated by reference in its entirety). G5-Gly-OH 2 was purified either by dialysis with a cellulose dialysis membrane against water, or buffer and then water. MTX and FA were attached to the hydroxyl groups of G5-Gly-OH 2 through ester bonds in a one-pot reaction with 2-chloro-1-methylpyridinium iodide and 4-(dimethylamino)pyridine as the coupling reagents (Mukaiyama reagent), which is efficient for preparation of esters from equimolar amounts of free carboxylic acid and alcohol. The reaction was conducted in dimethyl sulfoxide under nitrogen at room temperature for 24 hours. The final product, conjugate 3, was purified by dialysis with a cellulose dialysis membrane against PBS buffer and then water. The G5-FA-MTX is highly water-soluble even with 5 FAs and 15 MTXs attached. It was also observed that the numbers of FA and MTX attached on the surface of the PAMAM dendrimer were easily and reproducibly adjusted by changing the amount of FA and MTX in the feed. The numbers of FA and MTX attached on the PAMAM dendrimer were calculated using results from MALDI-TOP, GPC, and ¹H NMR.

¹H NMR

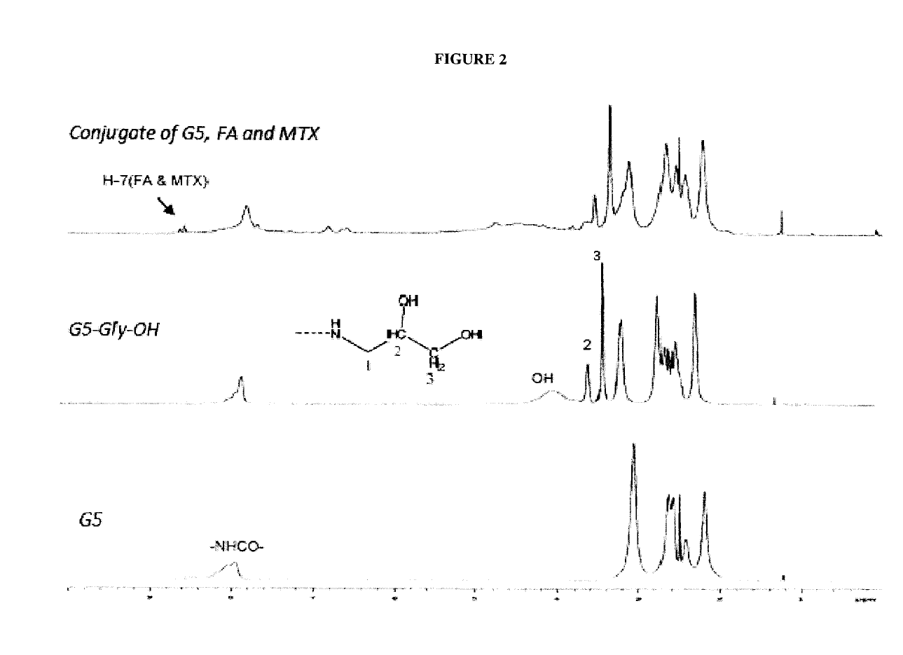

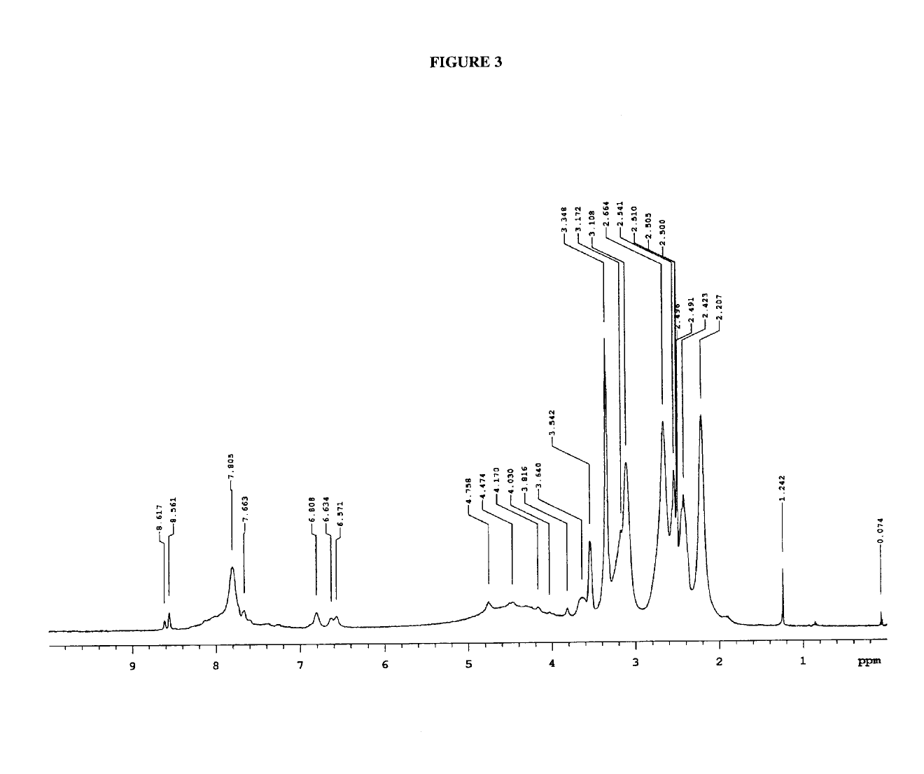

¹H NMR was used to confirm the surface capping of the terminal amines of the dendrimer with glycidol and attachment of FA and MTX to the hydroxyl group of the G5-Gly-OH (FIG. 2). The ¹H NMR of G5 PAMAM dendrimer shows 6 broad peaks which correspond to the protons of the amide bond (CONH) at 7.95 ppm, amino groups (NH₂), internal methylene groups (CH₂) and those CH₂ next to amino groups at 3.05, 2.64, 2.58, 2.42, and 2.20 ppm. The ¹H NMR spectrum of G5-Gly-OH clearly shows three new peaks compared with that of G5-NH₂. The peak at 3.348 ppm (peak 3) is the resonance of protons of CH₂ at position 3 of the 2,3-dihydroxylpropyl group. The peak at 3.542 ppm (peak 2) belongs to CH at position 2 of the 2,3-dihydroxylpropyl group. The broad peak at 3.961 ppm is attributed to two hydroxyl groups of the 2,3-dihydroxylpropyl group. The peak at about 2.47 ppm (24) belongs to the protons of CH₂ at position 1 of the 2,3-dihydroxylpropyl group, which overlaps with peaks associated with the internal protons of the G5-NH₂. Compared with the spectrum of G5-Gly-OH, the spectrum of G5-FA-MTX shows additional peaks. Some of the peaks overlap with the peaks of G5-Gly-OH. The H-7 peaks of conjugated FA and MTX are present at 8.617 ppm and 8.561 ppm, respectively. They are sharp and well separated single peaks. The integration ratio of these two peaks represents the molar ratio of MTX and FA because both MTX and FA contain only one H-7. Significantly, it is also found that the integration ratio of these two protons is proportional to the feed ratio of MTX and FA in the ‘one-pot’ reaction (Table 1). Therefore, the actual numbers of FA and MTX conjugated to the surface of the dendrimer can be calculated using this integration ratio and molecular weight gain from G5-Gly-OH 2 to G5-FA-MTX 3, as measured by MALDI-TOF and GPC. An ¹H NMR Spectrum of conjugate of G5 PAMAM dendrimer, FA, and MTX is shown in FIG. 3.

TABLE 1
Reaction feed molar ratio of MTX/FA/G5-Gly-OH, ¹H NMR
integration ratio of H-7 of MTX/FA, and Numbers of MTX and FA
on the conjugate (G5-FA-MTX)

| | ¹H NMR | Actual average |
|---|---|---|
| Reaction feed molar ratio | Integration Ratio of | number of MTX and |
| of MTX/FA/G5-Gly-OH | H-7s (MTX/FA) | FA attached on PAMAM |
| 10:7:1 | 1.38:1.00 | 6.79 and 5.20 |
| 15:8:1 | 1.86:1.00 | 9.44 and 5.22 |
| 23:8:1 | 2.83:1.00 | 14.90 and 5.42 |
| 15:10:1^{a} | 1.33:1.00 | 9.26 (±0.28^{b}) and |
| | | 7.18 (±0.1^{c}) |

^{a}Three batches of parallel synthesis under the same condition: G5-Gly-OH (50 mg), MTX (8.54 mg), FA (5.52 mg), 2-chloro-1-methylpyridinium iodide (8.00 mg), 4-(dimethylamino)pyridine (7.65 mg), DMSO (2.5 mL), N₂, 24 hours, cellulose membrane dialysis (6 times 4 L over 48 hours), and lyophilization (72 hours).
^{b}Standard deviation of average number of MTX.
^{c}Standard deviation of average number of FA.

MALDI-TOF

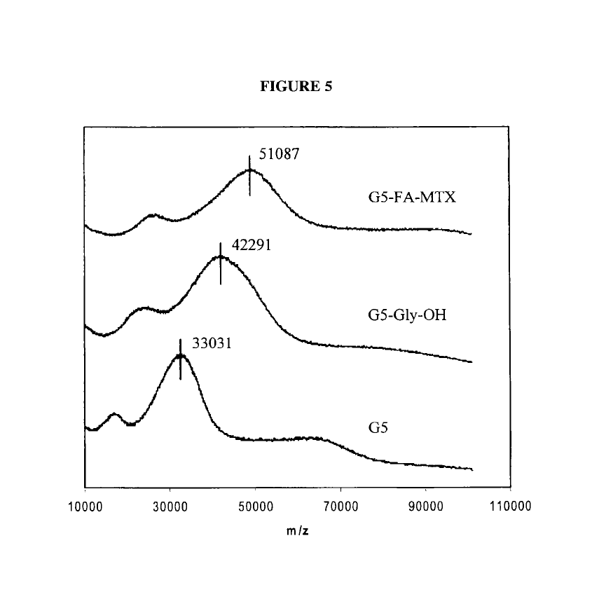

MALDI-TOF mass spectrometry has been proven to be an important technique for characterization of dendrimers and dendrimer derivatives. It not only provides molecular weight, but also gives information about the success of each conjugation reaction and the number of molecules that have been conjugated. FIG. 5 shows the MALDI-TOF mass spectra of G5-NH₂, G5-Gly-OH, and G5-FA-MTX. The molecular weight increase for each species along the synthetic pathway is clearly seen and demonstrates that the hydroxylation (first step) and conjugation (second step: ‘one pot’ reaction) have occurred. The molecular weight gain of G5-Gly-OH from G5-NH₂ is due to the attachment of the 2,2-dihydroxylpropyl group. The average number of 2,2-dihydroxylpropyl groups attached to each amino group may be calculated by the difference divided by the molecular mass of the 2,2-dihydroxylpropyl group and number of the amino groups on the surface of the dendrimer. The molecular weight increase from G5-Gly-OH to the G5-FA-MTX is due to the addition of both FA and MTX. The number of FAs and MTXs attached to the dendrimer can be calculated using formula (1) and (2), which are based on the molecular weight gain from G5-Gly-OH to G5-FA-MTX, as measured by MALDI-TOF or GPC, and the integration ratio of H-7 of conjugated FA and MTX. Calculation results have been listed in Table 1 (above).

$$\begin{matrix}{N}_{FA} = \frac{Δ MW \times {F}_{FA / ( MTX + FA )}}{{MW}_{FA} - 18} & ( 1 ) \\ {N}_{MTX} = \frac{Δ MW \times {F}_{MTX / ( MTX + FA )}}{{MW}_{MTX} - 18} & ( 2 )\end{matrix}$$

Note: N_{FA}: molecules of folic acid attached to each PAMAM dendrimer molecule; N_{MTX}: molecules of MTX attached to each PAMAM dendrimer molecule; ΔMW: molecular weight gain of conjugate from hydroxyl-terminated PAMAM dendrimer; F_{FA}: ¹H NMR integration fraction of H-7 (FA over FA and MTX); F_{MTX}: ¹H NMR integration fraction of H-7 (MTX over FA and MTX). MW_{FA}: molecular weight of FA; MW_{MTX}: molecular weight of MTX.

High Performance Liquid Chromatography (HPLC)

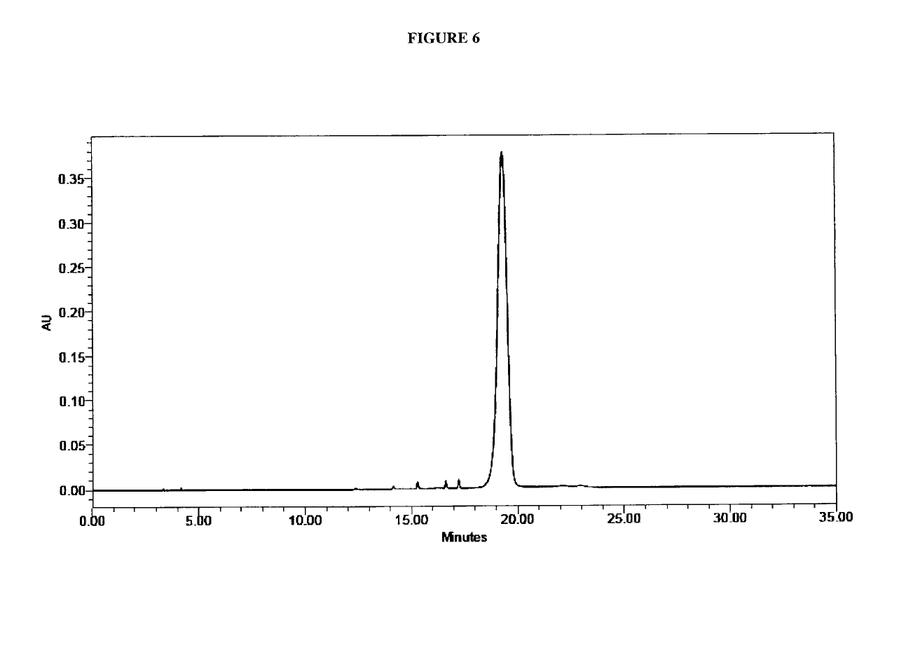

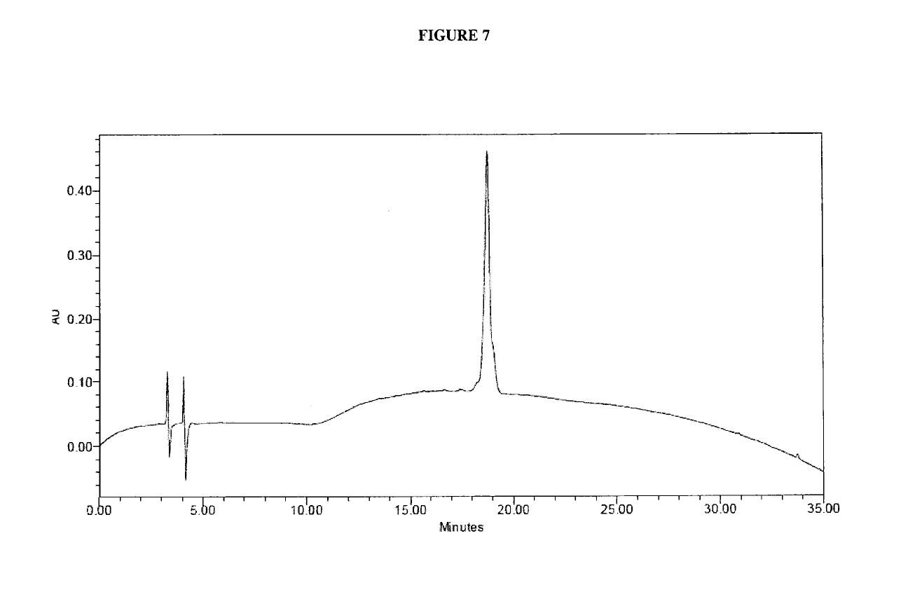

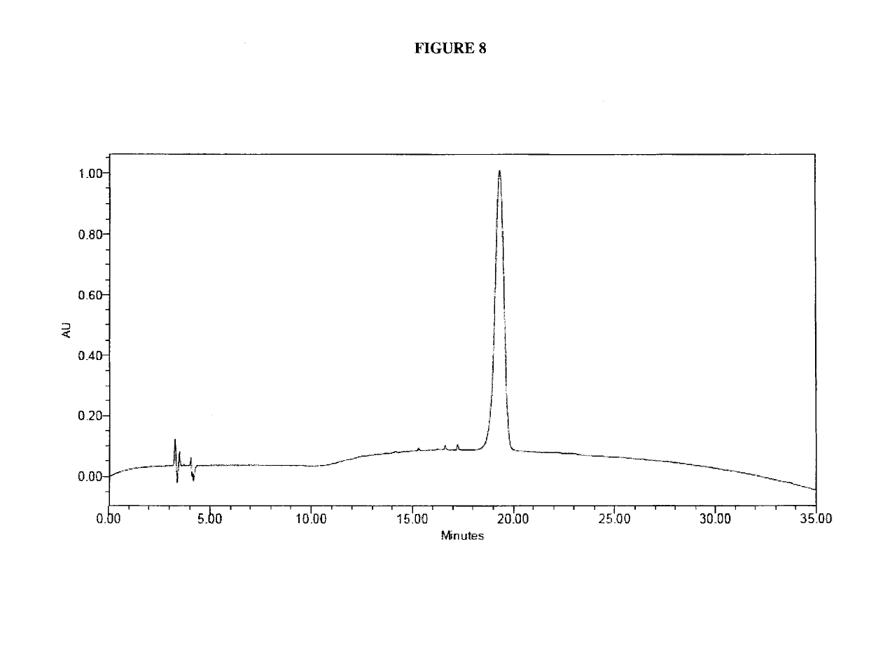

High performance liquid chromatography (HPLC) is a widely accepted method for separation and purification of small molecules and macromolecules and is commonly used in chemical, pharmaceutical, and biotech laboratories. Recently, HPLC has been used as a tool to analyze PAMAM dendrimers of various generations and terminal groups, as well as their conjugates (Islam et al. (2005) Analyt. Chem. 77:2063-2070; Islam et al. (2005) J. Chromatog. B-Analyt. Technol. Biomed. Life Sci. 822:21-26; Shi et al. (2006) Analyst 131:842-848; each herein incorporated by reference in its entirety). HPLC was used to evaluate the purity and molecular weight distributions of conjugates. Small molecule impurities such as unreacted FA, MTX, coupling reagent, and by-products are very easily differentiated from the dendrimers (G5-NH₂, G5-Gly-OH, G5-FA-MTX) by HPLC due to the significant difference in retention time. The polydispersity of PAMAM dendrimers can also be estimated by assessing the peak width at half height (W_{H/2}) (Islam et al. (2005) Analyt. Chem. 77:2063-2070; herein incorporated by reference in its entirety). Note that the polydispersity calculated from the W_{H/2} in HPLC chromatography is different from the one calculated from GPC experiments, which is the ratio of M_{w} and Mₙ. FIG. 6 is a representative chromatogram of the conjugate (G5-FA-MTX) under UV 280 nm which shows a single symmetric peak of the conjugate and a few small impurity peaks attributed to the presence of less than 0.1% free FA, MTX and other impurities. The result also indicates that the FA and MTX have been successfully attached to G5-Gly-OH resulting in a very narrow molecular weight distribution, which is in agreement with the GPC results shown below. An HPLC chromatogram of hydroxyl-terminated G5 PAMAM dendrimer under 210 nm is shown in FIG. 7. An HPLC chromatogram of hydroxyl-terminated G5 PAMAM dendrimer under 210 nm is shown in FIG. 8.

Gel Permeation Chromatography (GPC)

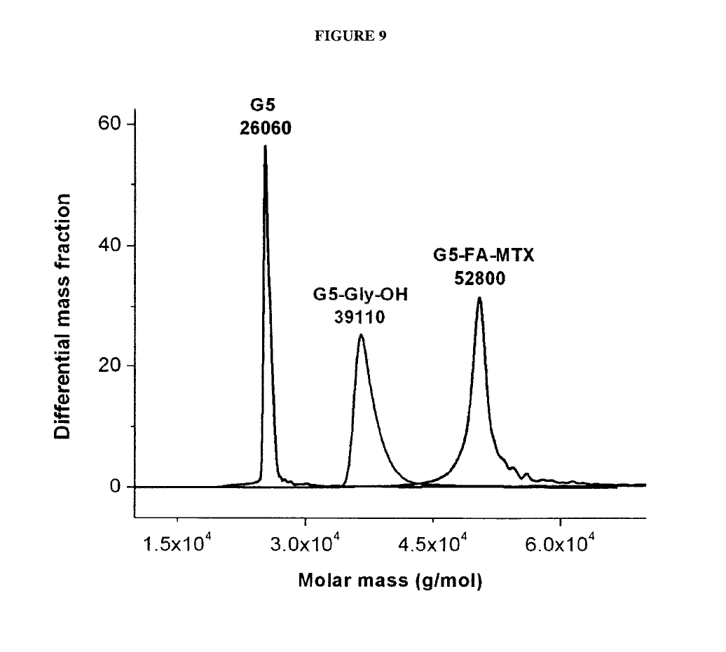

A gel permeation chromatography (GPC) instrument equipped with two detectors, a multiangle laser light scattering (MALLS) and a differential refractive index, was used to evaluate the molar mass of the starting PAMAM dendrimer, hydroxyl-terminated PAMAM dendrimer, and conjugates. FIG. 9 shows that differential mass fraction profile of G5-NH₂, G5-Gly-OH, and G5-FA-MTX respectively. All dendrimer samples exhibit a single peak. The number average molecular weights Mₙ and polydispersity index (PDI: M_{w}/Mₙ) of the G5-NH₂, G5-Gly-OH, and G5-FA-MTX are 26,060 (1.025), 39,110 (1.042), and 52,800 (1.044) respectively. The ratios of weight- to number-average molecular weights of the G5-NH₂, G5-Gly-OH, and G5-FA-MTX are very close to 1. This indicates that the distribution mass of the individual components is narrow and that the conjugations process at each step is nearly uniform.

The GPC results are somewhat different from those measured by MALDI-TOF, as shown by FIG. 5 and FIG. 9. This difference is due to the broadening of the MALDI-TOF spectra, as compared to the GPC spectra. Generally, however, GPC is thought to reflect the real average molar mass.

In Vitro Studies

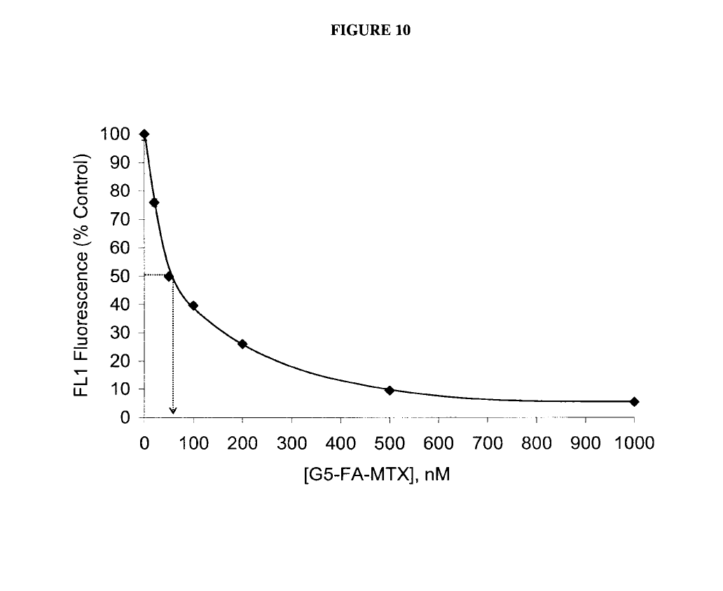

The binding of the newly synthesized G5-FA-MTX on to FA receptor expressing KB cells was examined A previously synthesized G5-FI-FA-MTX conjugate was utilized in which the FA and MTX were conjugated to the dendrimer through the classic synthetic pathway (Majoros et al. (2005) J. Med. Chem. 48:5892-5899; herein incorporated by reference in its entirety). In addition, a fluorescent dye FITC (FI) was conjugated to the surface of the dendrimer. As shown in FIG. 10, at a fixed concentration of G5-FI-FA-MTX (100 nM), the G5-FA-MTX competed with the former for binding, with a 50% inhibition of binding occurring at ˜60 nM. This shows that the G5-FA-MTX binds through the FA receptor with an affinity similar or slightly better than the G5-FA-MTX synthesized through the classic synthetic pathway (Majoros et al. (2005) J. Med. Chem. 48:5892-5899; herein incorporated by reference in its entirety).

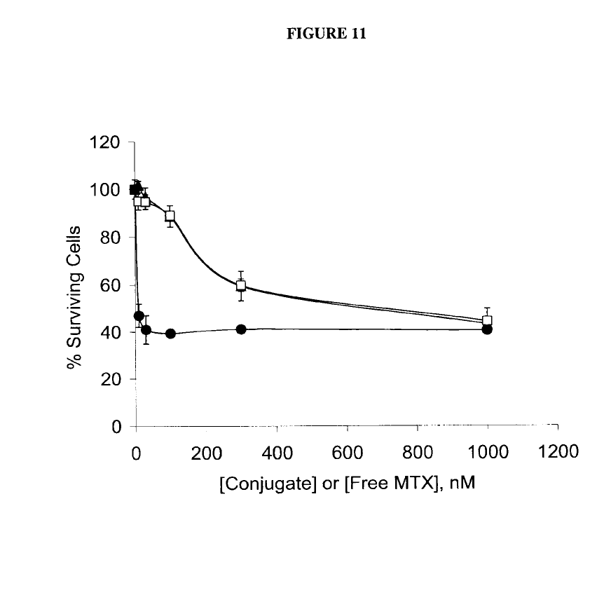

The cytotoxicity of the newly synthesized conjugate was determined by the XTT assay. The cytotoxicity was compared with another batch of G5-FA-MTX synthesized by Cambrex Inc. using the classic synthetic pathway in which the FA and MTX were conjugated through amide and ester linkages, respectively. Previous studies have shown that this batch of Cambrex conjugates was cytotoxic in vitro, and tumoricidal in vivo, with similar potency as that of our previously published compound (Kukowska-Latallo et al. (2005) Cancer Res. 65:5317-5324; herein incorporated by reference in its entirety). As shown in FIG. 11, the newly synthesized conjugate was as cytotoxic as the Cambrex batch. It was also found that both G5-FA-MTX and G5-MTX were cytotoxic in KB cells (FIG. 12). Notably, G5-MTX-FL, without having the traditional targeting ligand FA, also displayed high cytotoxicity.

Therefore, in experiments conducted during the course of developing some embodiments of the present invention, a novel targeted nanodendrimeric anticancer prodrug comprising a conjugate of PAMAM dendrimer, FA, and MTX was successfully synthesized using a simple ‘one pot’ procedure, which is reproducible and feasible for large scale synthesis because of the simple synthetic reaction and easy purification process. Using this method, FA and MTX can be attached to the dendrimer with any desired ratios in a simple one-step reaction. The final conjugate and all the intermediate products were characterized by ¹H NMR, MALDI-TOF, GPC, and HPLC. A new analytical approach for calculating molecules of FA and MTX attached to each dendrimer molecule was established. In vitro data show that the new conjugates have a similar cytotoxicity profile to previous batches of conjugates synthesized using the classical multi-step approach.

### Example 10

### Uptake of G5-FA-MTX-FL and G5-FA-FL by FR-Expressing KB Cells

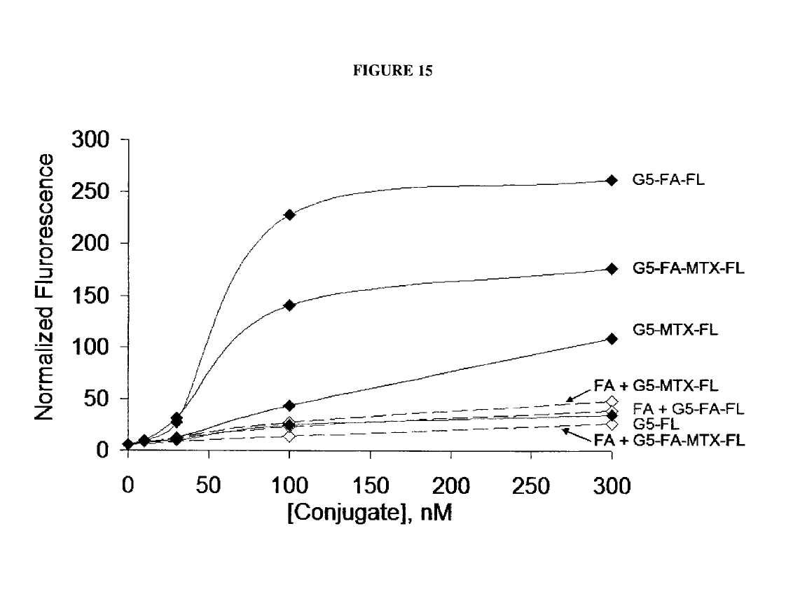

An in vitro study of polyvalent tumor-targeted conjugates of G5 PAMAM dendrimer with FA as the targeting agent, MTX as the chemotherapeutic drug, and fluorescein-5(6)-carboxamidohexanoic acid (FL) as the detecting agent was performed in FR-expressing KB cells. The result in FIG. 15 shows that both conjugate of G5 dendrimer, FA, and MTX (G5-FA-MTX) and conjugate of G5 dendrimer and MTX (G5-MTX) have affinity to FRs and displayed a high uptake rate, but G5-MTX exhibited a lower uptake pattern. While the present invention is not limited to any particular mechanism, and an understanding of the mechanism is not necessary to practice the present invention, it is contemplated that such uptake patterns are due to structural familiarity of FA and MTX.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1.-69. (canceled)

70. A method for synthesizing functionalized dendrimer nanodevices comprising simultaneous exposure of at least two different ligands to a dendrimer comprising terminal —OH groups, wherein said exposure occurs via a one-pot synthesis reaction.

71. The method of claim 70, wherein said dendrimer bearing terminal —OH groups comprises 2,3-dihydroxylpropyl groups.

72. The method of claim 70, wherein said exposure is conducted in the presence of ester coupling agents, wherein said ester coupling agents are selected from the group consisting of 2-chloro-1-methylpyridinium iodide in combination with 4-(dimethylamino)pyridine, dicyclohexylcarbodiimide in combination with 4-(dimethylamino)pyridine, other carbodiimide coupling agent in combination with 4-(dimethylamino)pyridine, and diethyl azodicarboxylate in combination with triphenylphosphine.

73. The method of claim 70, wherein said ligands are selected from the group consisting of therapeutic agents, targeting agents, trigger agents, and imaging agents.

74. The method of claim 70,
wherein said therapeutic agents are selected from the group consisting of chemotherapeutic agents, anti-oncogenic agents, anti-angiogenic agents, tumor suppressor agents, anti-microbial agents, expression constructs comprising a nucleic acid encoding a therapeutic protein, pain relief agents, pain relief agent antagonists, agents designed to treat arthritis, agents designed to treat inflammatory bowel disease, agents designed to treat an autoimmune disease, and agents designed to treat inflammatory pelvic disease;
wherein said targeting agents are selected from the group consisting of an agent binding a receptor selected from the group consisting of CFTR, EGFR, estrogen receptor, FGR2, folate receptor, IL-2 receptor, and VEGFR; an antibody that binds to a polypeptide selected from the group consisting of p53, Muc1, a mutated version of p53 that is present in breast cancer, HER-2, T and Tn haptens in glycoproteins of human breast carcinoma, and MSA breast carcinoma glycoprotein; an antibody selected from the group consisting of human carcinoma antigen, TP1 and TP3 antigens from osteocarcinoma cells, Thomsen-Friedenreich (TF) antigen from adenocarcinoma cells, KC-4 antigen from human prostrate adenocarcinoma, human colorectal cancer antigen, CA125 antigen from cystadenocarcinoma, DF3 antigen from human breast carcinoma, and p97 antigen of human melanoma, carcinoma or orosomucoid-related antigen; transferrin; and a synthetic tetanus toxin fragment;
wherein said trigger agents have functions selected from the group consisting of permitting a delayed release of a functional group from the dendrimer, permitting a constitutive release of the therapeutic agent from the dendrimer, permitting a release of a functional group from the dendrimer under conditions of acidosis, permitting a release of a functional group from a dendrimer under conditions of hypoxia, and permitting a release of the therapeutic agent from a dendrimer in the presence of a brain enzyme;
wherein said imaging agents are selected from the group consisting of fluorescein isothiocyanate (FITC), fluorescein-5(6)-carboxamidohexanoic acid, 6-TAMARA, acridine orange, and cis-parinaric acid.

75. The method of claim 70, wherein said dendrimer provides a linker with at least one terminal —OH group.

76. The method of claim 70, wherein at least one of said ligands provides a linker.

77. The method of claim 70, further comprising providing a linker.

78. The method of claim 70, wherein said at least two different ligands are selected from the group consisting of folic acid, methotrexate, SN-38, and fluorescein-5(6)-carboxamidocaproic acid.

79. The method of claim 70, wherein said at least two different ligands are selected from the group consisting of targeting agents, drugs or prodrugs, drug derivatives, and imaging agents comprising one or more carboxyl groups.

80. The method of claim 70, wherein said at least two different ligands have at least one carboxyl group.

81. The method of claim 70, wherein said exposure occurs in a suitable solvent system.

82. The method of claim 81, wherein said suitable solvent system comprises a polar solvent.

83. The method of claim 81, wherein said suitable solvent system comprises dimethylsulfoxide; N,N-dimethyl formamide; N-N-dimethylacetamide; dioxane; 2-pyrrolidinone; 1-methyl-2-pyrrolidinone or combination thereof.

84. The method of claim 70, wherein said dendrimer is derived from a PAMAM dendrimer.

85. The method of claim 84, wherein said PAMAM dendrimer derivative is generation 3 or greater.

86. The method of claim 70, further comprising purification of said functionalized dendrimer prior to inclusion in additional reactions, prior to analysis, or prior to final use.

87. The method of claim 86, wherein said purification is achieved by methods selected from the group consisting of dialysis and precipitation.

88. A composition comprising at least two different ligands, each attached to a dendrimer by an ester bond.

89. A method for treating a disorder comprising administering to a subject suffering from the disorder a composition as provided in claim 88, wherein said disorder is selected from the group consisting of any type of cancer or cancer-related disorder, a neoplastic disease, osteoarthritis, rheumatoid arthritis, septic arthritis, gout and pseudo-gout, juvenile idiopathic arthritis, psoriatic arthritis, Still's disease, and ankylosing spondylitis.


SEQUENCE LISTING (2 sequences)

SEQ ID NO: 1 - protein, 7 aa, Artificial Sequence
  Synthetic
YEVDGWK

SEQ ID NO: 2 - protein, 12 aa, Artificial Sequence
  Synthetic
HLNILSTLWKYR